SEQUENCE LISTING (5 sequences)

SEQ ID NO: 1 - protein, 1172 aa, Homo sapiens
MVWRLVLLALWVWPSTQAGHQDKDTTFDLFSISNINRKTIGAKQFRGPDPGVPAYRFVRFDYIPPVNADDLSKITKIMRQKEGFFLTAQLKQDGKSRGTLLALEGPGLSQRQFEIVSNGPADTLDLTYWIDGTRHVVSLEDVGLADSQWKNVTVQVAGETYSLHVGCDLIDSFALDEPFYEHLQAEKSRMYVAKGSARESHFRGLLQNVHLVFENSVEDILSKKGCQQGQGAEINAISENTETLRLGPHVTTEYVGPSSERRPEVCERSCEELGNMVQELSGLHVLVNQLSENLKRVSNDNQFLWELIGGPPKTRNMSACWQDGRFFAENETWVVDSCTTCTCKKFKTICHQITCPPATCASPSFVEGECCPSCLHSVDGEEGWSPWAEWTQCSVTCGSGTQQRGRSCDVTSNTCLGPSIQTRACSLSKCDTRIRQDGGWSHWSPWSSCSVTCGVGNITRIRLCNSPVPQMGGKNCKGSGRETKACQGAPCPIDGRWSPWSPWSACTVTCAGGIRERTRVCNSPEPQYGGKACVGDVQERQMCNKRSCPVDGCLSNPCFPGAQCSSFPDGSWSCGSCPVGFLGNGTHCEDLDECALVPDICFSTSKVPRCVNTQPGFHCLPCPPRYRGNQPVGVGLEAAKTEKQVCEPENPCKDKTHNCHKHAECIYLGHFSDPMYKCECQTGYAGDGLICGEDSDLDGWPNLNLVCATNATYHCIKDNCPHLPNSGQEDFDKDGIGDACDDDDDNDGVTDEKDNCQLLFNPRQADYDKDEVGDRCDNCPYVHNPAQIDTDNNGEGDACSVDIDGDDVFNERDNCPYVYNTDQRDTDGDGVGDHCDNCPLVHNPDQTDVDNDLVGDQCDNNEDIDDDGHQNNQDNCPYISNANQADHDRDGQGDACDPDDDNDGVPDDRDNCRLVFNPDQEDLDGDGRGDICKDDFDNDNIPDIDDVCPENNAISETDFRNFQMVPLDPKGTTQIDPNWVIRHQGKELVQTANSDPGIAVGFDEFGSVDFSGTFYVNTDRDDDYAGFVFGYQSSSRFYVVMWKQVTQTYWEDQPTRAYGYSGVSLKVVNSTTGTGEHLRNALWHTGNTPGQVRTLWHDPRNIGWKDYTAYRWHLTHRPKTGYIRVLVHEGKQVMADSGPIYDQTYAGGRLGLFVFSQEMVYFSDLKYECRDI

SEQ ID NO: 2 - protein, 1114 aa, Homo sapiens
MVWRLVLLALWVWPSTQAGHQDKDTTFDLFSISNINRKTIGAKQFRGPDPGVPAYRFVRFDYIPPVNADDLSKITKIMRQKEGFFLTAQLKQDGKSRGTLLALEGPGLSQRQFEIVSNGPADTLDLTYWIDGTRHVVSLEDVGLADSQWKNVTVQVAGETYSLHVGCDLIDSFALDEPFYEHLQAEKSRMYVAKGSARESHFRGLLQNVHLVFENSVEDILSKKGCQQGQGAEINAISENTETLRLGPHVTTEYVGPSSERRPEVCERSCEELGNMVQELSGLHVLVNQLSENLKRVSNDNQFLWELIGGPPKTRNMSACWQDGRFFAENETWVVDSCTTCTCKKFKTICHQITCPPATCASPSFVEGECCPSCLHSVDGEEGWSPWAEWTQCSVTCGSGTQQRGRSCDVTSNTCLGPSIQTRACSLSKCDTRIRQDGGWSHWSPWSSCSVTCGVGNITRIRLCNSPVPQMGGKNCKGSGRETKACQGAPCPNGCLSNPCFPGAQCSSFPDGSWSCGSCPVGFLGNGTHCEDLDECALVPDICFSTSKVPRCVNTQPGFHCLPCPPRYRGNQPVGVGLEAAKTEKQVCEPENPCKDKTHNCHKHAECIYLGHFSDPMYKCECQTGYAGDGLICGEDSDLDGWPNLNLVCATNATYHCIKDNCPHLPNSGQEDFDKDGIGDACDDDDDNDGVTDEKDNCQLLFNPRQADYDKDEVGDRCDNCPYVHNPAQIDTDNNGEGDACSVDIDGDDVFNERDNCPYVYNTDQRDTDGDGVGDHCDNCPLVHNPDQTDVDNDLVGDQCDNNEDIDDDGHQNNQDNCPYISNANQADHDRDGQGDACDPDDDNDGVPDDRDNCRLVFNPDQEDLDGDGRGDICKDDFDNDNIPDIDDVCPENNAISETDFRNFQMVPLDPKGTTQIDPNWVIRHQGKELVQTANSDPGIAVGFDEFGSVDFSGTFYVNTDRDDDYAGFVFGYQSSSRFYVVMWKQVTQTYWEDQPTRAYGYSGVSLKVVNSTTGTGEHLRNALWHTGNTPGQVRTLWHDPRNIGWKDYTAYRWHLTHRPKTGYIRVLVHEGKQVMADSGPIYDQTYAGGRLGLFVFSQEMVYFSDLKYECRDI

SEQ ID NO: 3 - protein, 787 aa, Homo sapiens
MVWRLVLLALWVWPSTQAGHQDKDTTFDLFSISNINRKTIGAKQFRGPDPGVPAYRFVRFDYIPPVNADDLSKITKIMRQKEGFFLTAQLKQDGKSRGTLLALEGPGLSQRQFEIVSNGPADTLDLTYWIDGTRHVVSLEDVGLADSQWKNVTVQVAGETYSLHVGCDLIDSFALDEPFYEHLQAEKSRMYVAKGSARESHFRGLLQNVHLVFENSVEDILSKKGCQQGQGAEINAISENTETLRLGPHVTTEYVGPSSERRPEVCERSCEELGNMVQELSGLHVLVNQLSENLKRVSNDNQFLWELIGGPPKTRNMSACWQDGRFFAENETWVVDSCTTCTCKKFKTICHQITCPPATCASPSFVEGECCPSCLHSVDGEEGWSPWAEWTQCSVTCGSGTQQRGRSCDVTSNTCLGPSIQTRACSLSKCDTRIRQDGGWSHWSPWSSCSVTCGVGNITRIRLCNSPVPQMGGKNCKGSGRETKACQGAPCPIDGRWSPWSPWSACTVTCAGGIRERTRVCNSPEPQYGGKACVGDVQERQMCNKRSCPVDGCLSNPCFPGAQCSSFPDGSWSCGSCPVGFLGNGTHCEDLDECALVPDICFSTSKVPRCVNTQPGFHCLPCPPRYRGNQPVGVGLEAAKTEKQVCEPENPCKDKTHNCHKHAECIYLGHFSDPMYKCECQTGYAGDGLICGEDSDLDGWPNLNLVCATNATYHCIKLWRPETQRQGASGAVLPLLLPASLAQASLGFWRWWLHGPDLWSASHHFLQCLSLRKLPLPLFDKNTCGCI

SEQ ID NO: 4 - protein, 334 aa, Homo sapiens
MVWRLVLLALWVWPSTQAGHQDKDTTFDLFSISNINRKTIGAKQFRGPDPGVPAYRFVRFDYIPPVNADDLSKITKIMRQKEGFFLTAQLKQDGKSRGTLLALEGPGLSQRQFEIVSNGPADTLDLTYWIDGTRHVVSLEDVGLADSQWKNVTVQVAGETYSLHVGCDLIDSFALDEPFYEHLQAEKSRMYVAKGSARESHFRGLLQNVHLVFENSVEDILSKKGCQQGQGAEINAISENTETLRLGPHVTTEYVGPSSERRPEVCERSCEELGNMVQELSGLHVLVNQLSENLKRVVGTHAGAAGVIGHGQLRRWPISAGINIPWLGSASCSM

SEQ ID NO: 5 - protein, 1095 aa, Homo sapiens
MRQKEGFFLTAQLKQDGKSRGTLLALEGPGLSQRQFEIVSNGPADTLDLTYWIDGTRHVVSLEDVGLADSQWKNVTVQVAGETYSLHVGCDLIDSFALDEPFYEHLQAEKSRMYVAKGSARESHFRGLLQNVHLVFENSVEDILSKKGCQQGQGAEINAISENTETLRLGPHVTTEYVGPSSERRPEVCERSCEELGNMVQELSGLHVLVNQLSENLKRVSNDNQFLWELIGGPPKTRNMSACWQDGRFFAENETWVVDSCTTCTCKKFKTICHQITCPPATCASPSFVEGECCPSCLHSVDGEEGWSPWAEWTQCSVTCGSGTQQRGRSCDVTSNTCLGPSIQTRACSLSKCDTRIRQDGGWSHWSPWSSCSVTCGVGNITRIRLCNSPVPQMGGKNCKGSGRETKACQGAPCPIDGRWSPWSPWSACTVTCAGGIRERTRVCNSPEPQYGGKACVGDVQERQMCNKRSCPVDGCLSNPCFPGAQCSSFPDGSWSCGSCPVGFLGNGTHCEDLDECALVPDICFSTSKVPRCVNTQPGFHCLPCPPRYRGNQPVGVGLEAAKTEKQVCEPENPCKDKTHNCHKHAECIYLGHFSDPMYKCECQTGYAGDGLICGEDSDLDGWPNLNLVCATNATYHCIKDNCPHLPNSGQEDFDKDGIGDACDDDDDNDGVTDEKDNCQLLFNPRQADYDKDEVGDRCDNCPYVHNPAQIDTDNNGEGDACSVDIDGDDVFNERDNCPYVYNTDQRDTDGDGVGDHCDNCPLVHNPDQTDVDNDLVGDQCDNNEDIDDDGHQNNQDNCPYISNANQADHDRDGQGDACDPDDDNDGVPDDRDNCRLVFNPDQEDLDGDGRGDICKDDFDNDNIPDIDDVCPENNAISETDFRNFQMVPLDPKGTTQIDPNWVIRHQGKELVQTANSDPGIAVGFDEFGSVDFSGTFYVNTDRDDDYAGFVFGYQSSSRFYVVMWKQVTQTYWEDQPTRAYGYSGVSLKVVNSTTGTGEHLRNALWHTGNTPGQVRTLWHDPRNIGWKDYTAYRWHLTHRPKTGYIRVLVHEGKQVMADSGPIYDQTYAGGRLGLFVFSQEMVYFSDLKYECRDI

(19) United States
(12) Patent Application Publication
(10) Pub. No.: US 2024/0255528 A1
(43) Pub. Date: Aug. 1, 2024

(54) FIBROSIS BIOMARKERS FOR NON-ALCOHOLIC FATTY LIVER DISEASE

(71) Applicant: The University of Hong Kong, Hong Kong (CN)

(72) Inventors: Chi-Ho LEE, Hong Kong (CN); Siu-Ling Karen LAM, Hong Kong (CN); Aimin XU, Hong Kong (CN)

(21) Appl. No.: 18/566,491

(22) PCT Filed: Jun. 1, 2021
(86) PCT No.: PCT/IB2021/054808

§ 371 Date: Dec. 1, 2023

(51) Int. Cl. G01N 33/68

(52) U.S. Cl. CPC G01N 33/6893; G01N 2800/085; G01N 2800/50

(57) ABSTRACT

Disclosed are biomarkers and methods for accurate non-invasive diagnosis or prognosis of liver fibrosis in subjects with non-alcoholic fatty liver disease (NAFLD). The biomarkers include circulating levels of thrombospondin 2 (TSP2) and can be measured at any time during the course of the disease. The methods include any one of non-invasive evaluation of circulating levels of TSP2 alone, or together with other molecular biomarkers, physiologic or radiographic biomarkers. The methods are highly sensitive and detect advanced liver fibrosis with a sensitivity of about or greater than 80%. The methods may use the circulating levels of TSP2 alone, or together with other molecular biomarkers, physiologic or radiographic biomarkers to accurately predict the risk of developing advanced liver fibrosis in a subject.

### FIELD OF THE INVENTION

This invention is generally directed to biomarkers and methods for non-invasive diagnosis or prognosis of fibrotic disease of the liver.

### BACKGROUND OF THE INVENTION

Thrombospondin (TSP) is a class of matricellular proteins that interacts with a number of ligands including extracellular matrix (ECM) structural proteins, cellular receptors, growth factors and cytokines. TSP modulates cell-matrix interactions and possesses anti-angiogenic properties. Among the five thrombospondins (TSP1-5), TSP1 and TSP2 share similar structure. Nonetheless, previous studies reported that TSP1 and TSP2 bind to different ligands and there are spatial and temporal differences in their expression, such that their roles are not interchangeable (Agah et al., Am J Pathol; 161:831-839 (2002); Helkin et al., Biochem Biophys Res Commun; 464:1022-1027 (2015); Zhang et al., Int J Mol Med; 45:1275-1293 (2020)).

Hyperglycemia could induce both TSP1 and TSP2 expression, and increased tissue expression of TSP1 and TSP2 had been found in patients with type 2 diabetes. With regard to non-alcoholic fatty liver disease (NAFLD), genetic inhibition of TSP1 was shown to protect mice from developing non-alcoholic steatohepatitis (NASH), and serum TSP1 levels were found to correlate positively with the degree of hepatic steatosis in patients with NAFLD (Min-DeBartolo et al., PLoS One; 14:e0226854 (2019); Bai et al., EBioMedicine; 57:102849 (2020)). A study revealed significant up-regulation of the hepatic expression of THBS2 gene, which encodes TSP2, in patients with advanced fibrosis compared to those without (Lou et al., Sci Rep; 7:4748 (2017)), yet the clinical relevance of circulating TSP2 is unknown.

Type 2 diabetes is an important risk factor of progression in NAFLD (Younossi et al., Clin Gastroenterol Hepatol; 2:262-265 (2004); Kim et al., Clin Gastroenterol Hepatol; 17:543-550 e542 (2019); and Zoppini et al., Am J Gastroenterol; 109:1020-1025 (2014)). Among the various stages in the spectrum of NAFLD, liver fibrosis is the major determinant of overall mortality and adverse liver-related outcomes (Angulo et al., Gastroenterology; 149:389-397 e310 (2015); Ekstedt et al., Hepatology; 61:1547-1554 (2015)). Strikingly, over 70% of patients with type 2 diabetes have concomitant NAFLD, or more specifically, metabolic dysfunction-associated fatty liver disease (MAFLD) using the recently proposed definition (Eslam et al., J Hepatol., 73:202-209 (2020)). NAFLD is the most prevalent chronic liver disease in the U.S. NAFLD exists as two predominant histological subtypes: nonalcoholic fatty liver (NAFL) and nonalcoholic steatohepatitis (NASH) (Kleiner et al., Hepatology.; 41(6):1313-1321 (2005)). NAFL is associated with a relatively benign clinical course, while NASH is associated with increased risk of progressive fibrosis and cirrhosis. NASH may be defined by the presence of hepatic steatosis and inflammation with hepatocyte injury (ballooning) with or without fibrosis (Chalasani et al., Hepatology; 55:2005-23 (2012)).

Non-alcoholic fatty liver disease (NAFLD) consists of a spectrum of hepatic disorders ranging from isolated hepatic steatosis, to non-alcoholic steatohepatitis (NASH), advanced fibrosis, cirrhosis and the development of hepatocellular carcinoma (HCC) (Chalasani et al. Diagnosis and Management of NAFLD: Practice Guidance from AASLD; Hepatology 2018). NAFLD can be diagnosed by the presence of hepatic steatosis on imaging OR histology, after exclusion of secondary causes of hepatic fat accumulation. NASH, however, is a histological diagnosis that can only be diagnosed with liver biopsy, which include the presence of inflammation with hepatocyte injury (ballooning) with and without fibrosis. Liver fibrosis can be assessed non-invasively using clinical decision aids (e.g., NAFLD fibrosis score), imaging (e.g., VTCE, MR elastography).

In NAFLD, liver biopsy remains the gold standard for histological diagnosis, assessing activity and staging fibrosis. However, routine use of liver biopsy is limited by its invasive nature, risk of complications, cost, sampling error, and poor patient acceptance. This underscores an urgent need for non-invasive and accurate methods for disease detection and staging. There are currently no reliable non-invasive means of differentiating NAFL from NASH (Siddiqui et al., Clin Gastroenterol Hepatol.; 17(1):156-163 (2019)). Also, some individuals with advanced fibrosis have relatively little NASH (Caldwell et al., Annals of Hepatology, 8, 346-352 (2009)).

Therefore, there is a pressing need for prognostic biomarkers to identify those who are at a higher risk of disease progression, in particular the development of advanced fibrosis, as these patients are at a higher risk of developing long-term liver-related morbidity and mortality (Lee et al., J Diabetes Investig; 8:131-133 (2017)).

Therefore, it is the object of the present invention to provide biomarkers for non-invasive detection of fibrosis and prognosis of fibrosis progression in NAFLD.

It is another object of the present invention to provide methods for non-invasive detection of fibrosis and prognosis of fibrosis progression in NAFLD.

### SUMMARY OF THE INVENTION

Described are methods for non-invasively detecting advanced liver fibrosis in a subject. Described are also methods for detecting risk of developing advanced liver fibrosis in a subject. The methods typically include measuring circulating levels of a biomarker thrombospondin 2 (TSP2) in a sample from the subject. The measuring of the circulating levels of TSP2 may be with a set of capture reagents containing one or more antibody binding fragments having a binding specificity to TSP2, preferably, to human TSP2. The set of capture reagents may include binding fragments having a binding specificity to TSP2 and no binding specificity to TSP1, TSP3, TSP4 and TSP5. Generally, the method detects circulating levels of TSP2 in nanogram/ml range, such as between 0.2 ng/ml and 10 ng/ml in a sample from a subject. The method of measuring may be an immunoassay having a lowest detection limit for TSP2 between about 0.156 ng/ml and about 1 ng/ml, such as between about 0.2 ng/ml and about 0.5 ng/ml, or about 0.5 ng/ml.

Generally, the method detects advanced liver fibrosis when the circulating levels of TSP2 in a sample from a subject are greater than about 3.6 ng/ml. Typically, the subject has non-alcoholic fatty liver disease (NAFLD). The subject may also have one or more other diseases or conditions, such as metabolic syndrome, type 2 diabetes mellitus, cardiovascular disease (CVD), and chronic kidney disease (CKD). The subjects may have NAFLD and type 2 diabetes. The subject may or may not have non-alcoholic steatohepatitis (NASH).

The method typically utilizes a blood or serum sample from the subject to measure the circulating levels of TSP2.

Typically, the method provides non-invasive detection of advanced liver fibrosis or non-invasive detection of risk of progression to grade F3 or higher liver fibrosis. Typically, grade F3 or higher advanced liver fibrosis is measured by vibration controlled transient elastography (VCTE). The advanced liver fibrosis of about or over grade F3 is fibrosis graded by liver stiffness (LS) measurement on VCTE having a cut off value of about 9.6 kiloPascal (kPa) on M probe or 9.3 kPa on XL probe, and greater.

The method typically detects advanced liver fibrosis with a sensitivity of about or greater than 80% and specificity of about or greater than 60% when the circulating levels of TSP2 in the sample from the subject are greater than about 3.6 ng/ml. The method typically detects advanced liver fibrosis with negative predictive value of about or over 90%.

Also described is a method of detecting risk of developing of advanced liver fibrosis over time in a subject. Typically, the method includes measuring circulating levels of a biomarker TSP2 in a sample from the subject. The method detects risk of developing of advanced liver fibrosis over time is 2.82 times greater per unit increase in the log-transformed serum TSP2 level measured in ng/ml. Typically, the period of time is between about 0.1 years and about 3 years from obtaining the sample, from measuring the circulating levels of TSP2, or both.

Also described are kits and immunoassays with a set of capture reagents containing one or more antibody binding fragments having a binding specificity to TSP2, preferably to human TSP2.

The use of circulating TSP2 level as a novel fibrosis biomarker of grade F3 or higher fibrosis in NAFLD enables early hepatic risk stratification among the large number of NAFLD patients, with and without concomitant type 2 diabetes. Patients with high circulating TSP2 level, which indicates a greater risk of harboring advanced fibrosis and fibrosis progression, can be identified for referrals to hepatologists for further evaluation, more vigilant surveillance for the development of adverse liver outcomes (cirrhosis, varices and liver cancer etc.). Moreover, these patients can be prioritized for anti-diabetic agents that could improve hepatic fibrosis, liver dysfunction and/or fat content, as well as new NAFLD treatments when clinically available, especially in places where health resource is limited.

### BRIEF DESCRIPTION OF THE DRAWINGS

FIG. 1 is a graph of receiver operating characteristic curves for the identification of ≥F3 fibrosis in study participants with and without the addition of circulating TSP2 level to clinical risk factors. Data shown are AUROC with their 95% CI in parentheses. AUROC, area under the receiver operating characteristic curve; TSP2, thrombospondin 2; BMI, body mass index; AST, aspartate aminotransferase.

### DETAILED DESCRIPTION OF THE INVENTION

### I. Definitions

As used herein, the term “advanced fibrosis” refers to fibrosis of the liver characterized by non-invasive detection using vibration controlled transient elastography as having grade F3 or higher grade fibrosis graded by LS cut-offs: F3 9.6-11.4 kPa and F4≥11.5 kPa (M Probe); F3 9.3-10.9 kPa and F4≥11.0 kPa (XL Probe) (Kwok et al., Gut; 65:1359-1368 (2016)).

As used herein, the term “biomarker” refers to a molecular, histologic, radiographic, and/or a physiologic characteristic that is measured as an indicator of normal biological processes, pathogenic processes, or responses to an exposure or intervention, including therapeutic interventions. The biomarker may be a diagnostic and/or or prognostic biomarker used for detecting a tissue state or disease, monitoring a tissue state or disease, and/or predicting tissue state or disease. For example, a measure of a biomolecule that is a biomarker may provide information about a tissue state as a diagnostic biomarker, as well as information about future changes in the tissue state as a prognostic biomarker. Other examples of biomarkers include body mass index (BMI) as a physiologic biomarker, or tissue elasticity as a radiographic biomarker, both of which may be diagnostic biomarkers and prognostic biomarkers.

As used herein, the term “non-invasive” or “non-invasively”, in the context of detecting, refers to a mode of obtaining information about an organ of interest without physically taking a sample from the organ of interest, such as without a biopsy of the organ of interest. For example, non-invasively detecting advanced liver fibrosis refers to detecting advanced liver fibrosis without biopsy of the liver.

As used herein, the term “antibody” refers to antibodies, such as polyclonal or monoclonal immunoglobulin molecules. In addition to intact immunoglobulin molecules, also included are fragments or polymers of those immunoglobulin molecules, and human or humanized versions of immunoglobulin molecules or fragments thereof, as long as the molecules maintain the ability to bind with an epitope, such as an epitope of TSP2. The antibodies can be tested for their desired activity using the in vitro assays, or by analogous methods, after which their in vivo therapeutic and/or diagnostic activities can be confirmed and quantified according to known clinical testing methods.

In some embodiments, the antibody is a monoclonal antibody or a binding fragment thereof. A monoclonal antibody refers to an antibody where individual antibodies within a population are identical.

As used herein, the term “isolated antibody” refers to an antibody which is substantially free of other antibodies having different antigenic specificities (e.g., an isolated antibody that specifically binds to TSP2, is substantially free of antibodies that specifically bind antigens other than TSP2). An isolated antibody specifically binds to an epitope, isoform or variant of TSP2. Moreover, an isolated antibody may be substantially free of other cellular material and/or chemicals.

As used herein, the terms “binding fragment,” “antigen binding fragment,” “antibody binding fragment,” and the like, refer to one or more portions of an antibody that contain the antibody's CDRs and, optionally, the framework residues that comprise the antibody's “variable region” antigen recognition site, and exhibit an ability to immunospecifically bind antigen. Such fragments include Fab′, F(ab′)2, Fv, single chain (ScFv), etc., and mutants and variants thereof, naturally occurring variants.

As used herein, the term “fragment” refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues.

The variable regions can also be substituted and altered in ways that do not eliminate the binding and binding specificity of the variable region or CDRs. For the disclosed antibodies and polypeptides with substitutions, alterations, eliminations, etc. of portions of antibodies other than the variable regions (or other than the CDRs), it is preferred that the variable region sequences and the CDR sequences are, or are modeled after, the variable regions or CDRs of disclosed monoclonal antibody.

As used herein, the terms “binding specificity,” “specificity,” “specifically reacts,” “specifically interacts,” or “specific to” refers to the ability of an antibody or other agent to detectably bind an epitope presented on an antigen, such as epitopes of TSP2, while having relatively little detectable reactivity with other structures. Specificity can be relatively determined by binding or competitive assays, using e.g., Biacore instruments. Specificity can be exhibited by, e.g., an about 5:1, about 10:1, about 20:1, about 50:1, about 100:1, about 10,000:1 or greater ratio of affinity/avidity in binding to the specific antigen versus nonspecific binding to other irrelevant molecules. In the context of the disclosed antibodies and polypeptides, “bi-specific” and similar terms refer to antibodies or polypeptides containing at least two different specific binding elements that each specifically binds to a different epitope or ligand.

As used herein, the term “detect”, “detecting”, “determine” or “determining” generally refers to obtaining information. Detecting or determining can utilize any of a variety of techniques available to those skilled in the art, including for example specific techniques explicitly referred to herein. Detecting or determining may involve manipulation of a physical sample, consideration and/or manipulation of data or information, for example utilizing a computer or other processing unit adapted to perform a relevant analysis, and/or receiving relevant information and/or materials from a source. Detecting or determining may also mean comparing an obtained value to a known value, such as a known test value, a known control value, or a threshold value. Detecting or determining may also mean forming a conclusion based on the difference between the obtained value and the known value.

As used herein, the term “sensitivity” refers to the ability of a test to correctly identify true positives, i.e., subjects with liver fibrosis For example, sensitivity can be expressed as a percentage, the proportion of actual positives which are correctly identified as such (e.g., the percentage of test subjects having liver fibrosis correctly identified by the test as having liver fibrosis). A test with high sensitivity has a low rate of false negatives, i.e., the cases with liver fibrosis not identified as such. Generally, the disclosed assays and methods have a sensitivity of at least about 80%, at least about 85%, at least about 90%, at least 92%, at least 94%, at least 95%, at least 96%, at least 97%, at least 98%, at least 99%, or at least 100%.

As used herein, the term “specificity” refers to the ability of a test to correctly identify true negatives, i.e., the subjects without liver fibrosis. For example, specificity can be expressed as a percentage, the proportion of actual negatives which are correctly identified as such (e.g., the percentage of test subjects not having liver fibrosis correctly identified by the test as not having liver fibrosis). A test with high specificity has a low rate of false positives, i.e., the cases of individuals not having liver fibrosis but suggested by the test as having liver fibrosis. Generally, the disclosed methods have a specificity of at least about 60%, at least about 70%, at least about 80%, at least about 90%, at least 92%, at least 94%, at least 95%, at least 96%, at least 97%, at least 98%, at least 99%, or at least 100%.

As used herein, the term “accurate” refers to the ability of a test to provide results with high sensitivity and high specificity, such as with sensitivity over about 80% and specificity over about 60%, with sensitivity over about 85% and specificity over about 65%, or with sensitivity over about 90% and specificity over about 80%.

As used herein, the term “sample” refers to body fluids, body smears, cell, tissue, organ or portion thereof that is isolated from a subject. A sample may be a single cell or a plurality of cells. A sample may be a specimen obtained by biopsy (e.g., surgical biopsy). A sample may be cells from a subject that are or have been placed in or adapted to tissue culture. A sample may be one or more of cells, tissue, serum, plasma, urine, spittle, sputum, and stool. A sample may be one or more of a saliva, sputum, tear, sweat, urine, exudate, blood, serum, plasma, or a vaginal discharge.

As used herein, the terms “subject,” “individual” or “patient” refer to a human or a non-human mammal. A subject may be a non-human primate, domestic animal, farm animal, or a laboratory animal. For example, the subject may be a dog, cat, goat, horse, pig, mouse, rabbit, or the like. The subject may be a human. The subject may be healthy, suffering from, or susceptible to a disease, disorder or condition. A patient refers to a subject afflicted with a disease or disorder. The term “patient” includes human and veterinary subjects.

A “control” sample or value refers to a sample that serves as a reference, usually a known reference, for comparison to a test sample. For example, a test sample can be taken from a test subject, and a control sample can be taken from a control subject, such as from a known normal (non-disease) individual. A control can also represent an average value gathered from a population of similar individuals, e.g., disease patients or healthy individuals with a similar medical background, same age, weight, etc. One of skill will recognize that controls can be designed for assessment of any number of parameters.

As used herein the terms “treatment” or “treating” refer to administering a composition to a subject or a system to treat one or more symptoms of a disease. The effect of the administration of the composition to the subject can be, but is not limited to, the cessation of a particular symptom of a condition, a reduction or prevention of the symptoms of a condition, a reduction in the severity of the condition, the complete ablation of the condition, a stabilization or delay of the development or progression of a particular event or characteristic, or minimization of the chances that a particular event or characteristic will occur.

As used herein the terms “effective amount” and “therapeutically effective amount,” used interchangeably, as applied to the nanoparticles, therapeutic agents, and pharmaceutical compositions described herein, refer to the quantity necessary to render the desired therapeutic result. For example, an effective amount is a level effective to treat, cure, or alleviate the symptoms of a disease for which the composition and/or therapeutic agent, or pharmaceutical composition, is/are being administered. Amounts effective for the particular therapeutic goal sought will depend upon a variety of factors including the disease being treated and its severity and/or stage of development/progression; the bioavailability and activity of the specific compound and/or antineoplastic, or pharmaceutical composition, used; the route or method of administration and introduction site on the subject.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%.

### II. Biomarker for Advanced Liver Fibrosis

Biomarkers for non-invasively detecting advanced liver fibrosis, or for determining risk of developing advanced liver fibrosis, in the subject include molecular, radiographic, and/or a physiologic biomarkers. The biomarkers may be used alone or in any combination. For example, any one or more of the molecular biomarkers may be used with any one or more of the radiographic biomarkers, and/or with any one or more physiologic biomarkers. In some embodiments, the one or more molecular biomarkers are used with one or more physiologic biomarkers, and/or with the one or more radiographic biomarkers, to detect or predict risk of developing advanced liver fibrosis.

Typically, the molecular biomarkers include TSP2 and aspartate transaminase (AST). Typically, the physiologic biomarker is body mass index (BMI, kg/m²). Typically, the radiographic biomarkers include liver stiffness (LS, kPa) and controlled attenuation parameter (CAP, dB/m) readings on vibration controlled transient elastography.

### A. Thrombospondin-2 in Liver Fibrosis

The TSP family contains five members (TSP1-5) that represent multimeric glycoproteins, which bind Ca²⁺, interact with other ECM proteins, and contribute to the associations between cells and between cells and ECM. The TSP family is divided to two subgroups: trimeric subgroup A (TSP1 and TSP2) and pentameric subgroup B (TSP3, TSP4, and TSP5). TSPs have a complex multidomain structure. The C-terminal domain, type III repeats and epidermal growth factor (EGF)-like repeats are present in all TSPs and underline the TSP family. The oligomerization domain can be also found in all family members but it is more variable compared with other shared structures. The subgroup A has three EGF-like repeats and type I repeats (also known as thrombospondin repeats; TSRs), von Willebrand factor type C (vWC) domains, and the N-terminal domain. The subgroup B contains four EGF-like repeats but vWC domains and TSRs are missing (Chistiakov et al., Int. J. Mol. Sci., 18, 1540:1-29 (2017)).

The TSP2 protein of exists in cell-bound form as well in a circulating form. TSP2 is a member of a group of functionally related extracellular matrix (ECM) glycoproteins, which can mediate extracellular matrix assembly, cell-to-matrix interactions, degradation of matrix metalloproteinase (MMP)-2 and MMP-9, and inhibition of angiogenesis. Besides angiogenesis, TSP2 has been reported to interact with multiple cell receptors, growth factors and ECM proteins as well as regulate apoptosis, cell proliferation and adhesion. The expression of TSP2 and its prognostic value have been investigated in several cancers (Tian et al., JBUON; 23(5):1331-1336 (2018)).

TSP2 is 150 kDa calcium-binding protein released from various types of cell. The amino acid sequence of human TSP2 is as follows.

NP_003238.2
(SEQ ID NO: 1)
MVWRLVLLALWVWPSTQAGHQDKDTTFDLFSISNINRKTIGAKQFRGPDP
GVPAYRFVRFDYIPPVNADDLSKITKIMRQKEGFFLTAQLKQDGKSRGTL
LALEGPGLSQRQFEIVSNGPADTLDLTYWIDGTRHVVSLEDVGLADSQWK
NVTVQVAGETYSLHVGCDLIDSFALDEPFYEHLQAEKSRMYVAKGSARES
HFRGLLQNVHLVFENSVEDILSKKGCQQGQGAEINAISENTETLRLGPHV
TTEYVGPSSERRPEVCERSCEELGNMVQELSGLHVLVNQLSENLKRVSND
NQFLWELIGGPPKTRNMSACWQDGRFFAENETWVVDSCTTCTCKKFKTIC
HQITCPPATCASPSFVEGECCPSCLHSVDGEEGWSPWAEWTQCSVTCGSG
TQQRGRSCDVISNICLGPSIQTRACSLSKCDTRIRQDGGWSHWSPWSSCS
VTCGVGNITRIRLCNSPVPQMGGKNCKGSGRETKACQGAPCPIDGRWSPW
SPWSACTVTCAGGIRERTRVCNSPEPQYGGKACVGDVQERQMCNKRSCPV
DGCLSNPCFPGAQCSSFPDGSWSCGSCPVGFLGNGTHCEDLDECALVPDI
CFSTSKVPRCVNTQPGFHCLPCPPRYRGNQPVGVGLEAAKTEKQVCEPEN
PCKDKTHNCHKHAECIYLGHFSDPMYKCECQTGYAGDGLICGEDSDLDGW
PNLNLVCATNATYHCIKDNCPHLPNSGQEDFDKDGIGDACDDDDDNDGVT
DEKDNCQLLFNPRQADYDKDEVGDRCDNCPYVHNPAQIDIDNNGEGDACS
VDIDGDDVFNERDNCPYVYNTDQRDTDGDGVGDHCDNCPLVHNPDQTDVD
NDLVGDQCDNNEDIDDDGHQNNQDNCPYISNANQADHDRDGQGDACDPDD
DNDGVPDDRDNCRLVFNPDQEDLDGDGRGDICKDDFDNDNIPDIDDVCPE
NNAISETDFRNFQMVPLDPKGITQIDPNWVIRHQGKELVQTANSDPGIAV
GFDEFGSVDFSGTFYVNTDRDDDYAGFVFGYQSSSRFYVVMWKQVTQTYW
EDQPTRAYGYSGVSLKVVNSTTGTGEHLRNALWHIGNTPGQVRILWHDPR
NIGWKDYTAYRWHLTHRPKTGYIRVLVHEGKQVMADSGPIYDQTYAGGRL
GLFVFSQEMVYFSDLKYECRDI
NP_001368868.1
(SEQ ID NO: 2)
MVWRLVLLALWVWPSTQAGHQDKDTTFDLFSISNINRKTIGAKQFRGPDP
GVPAYRFVREDYIPPVNADDLSKITKIMRQKEGFFLTAQLKQDGKSRGTL
LALEGPGLSQRQFEIVSNGPADTLDLTYWIDGTRHVVSLEDVGLADSQWK
NVTVQVAGETYSLHVGCDLIDSFALDEPFYEHLQAEKSRMYVAKGSARES
HFRGLLQNVHLVFENSVEDILSKKGCQQGQGAEINAISENTETLRLGPHV
TTEYVGPSSERRPEVCERSCEELGNMVQELSGLHVLVNQLSENLKRVSND
NQFLWELIGGPPKTRNMSACWQDGRFFAENETWVVDSCTTCTCKKFKTIC
HQITCPPATCASPSFVEGECCPSCLHSVDGEEGWSPWAEWTQCSVTCGSG
TQQRGRSCDVISNTCLGPSIQTRACSLSKCDTRIRQDGGWSHWSPWSSCS
VTCGVGNITRIRLCNSPVPQMGGKNCKGSGRETKACQGAPCPNGCLSNPC
FPGAQCSSFPDGSWSCGSCPVGFLGNGTHCEDLDECALVPDICFSTSKVP
RCVNTQPGFHCLPCPPRYRGNQPVGVGLEAAKTEKQVCEPENPCKDKTHN
CHKHAECIYLGHFSDPMYKCECQTGYAGDGLICGEDSDLDGWPNLNLVCA
TNATYHCIKDNCPHLPNSGQEDFDKDGIGDACDDDDDNDGVTDEKDNCQL
LFNPRQADYDKDEVGDRCDNCPYVHNPAQIDIDNNGEGDACSVDIDGDDV
FNERDNCPYVYNTDQRDTDGDGVGDHCDNCPLVHNPDQTDVDNDLVGDQC
DNNEDIDDDGHQNNQDNCPYISNANQADHDRDGQGDACDPDDDNDGVPDD
RDNCRLVFNPDQEDLDGDGRGDICKDDFDNDNIPDIDDVCPENNAISETD
FRNFQMVPLDPKGTTQIDPNWVIRHQGKELVQTANSDPGIAVGFDEFGSV
DFSGTFYVNTDRDDDYAGFVFGYQSSSRFYVVMWKQVTQTYWEDQPTRAY
GYSGVSLKVVNSTIGTGEHLRNALWHIGNTPGQVRILWHDPRNIGWKDYT
AYRWHLTHRPKTGYIRVLVHEGKQVMADSGPIYDQTYAGGRLGLFVFSQE
MVYFSDLKYECRDI
NP_001368869.1
(SEQ ID NO: 3)
MVWRLVLLALWVWPSTQAGHQDKDTTFDLFSISNINRKTIGAKQFRGPDP
GVPAYRFVRFDYIPPVNADDLSKITKIMRQKEGFFLTAQLKQDGKSRGTL
LALEGPGLSQRQFEIVSNGPADTLDLTYWIDGTRHVVSLEDVGLADSQWK
NVTVQVAGETYSLHVGCDLIDSFALDEPFYEHLQAEKSRMYVAKGSARES
HFRGLLQNVHLVFENSVEDILSKKGCQQGQGAEINAISENTETLRLGPHV
TTEYVGPSSERRPEVCERSCEELGNMVQELSGLHVLVNQLSENLKRVSND
NQFLWELIGGPPKTRNMSACWQDGRFFAENETWVVDSCTTCTCKKFKTIC
HQITCPPATCASPSFVEGECCPSCLHSVDGEEGWSPWAEWTQCSVTCGSG
TQQRGRSCDVTSNTCLGPSIQTRACSLSKCDTRIRQDGGWSHWSPWSSCS
VTCGVGNITRIRLCNSPVPQMGGKNCKGSGRETKACQGAPCPIDGRWSPW
SPWSACTVTCAGGIRERTRVCNSPEPQYGGKACVGDVQERQMCNKRSCPV
DGCLSNPCFPGAQCSSFPDGSWSCGSCPVGFLGNGTHCEDLDECALVPDI
CFSTSKVPRCVNTQPGFHCLPCPPRYRGNQPVGVGLEAAKTEKQVCEPEN
PCKDKTHNCHKHAECIYLGHFSDPMYKCECQTGYAGDGLICGEDSDLDGW
PNLNLVCATNATYHCIKLWRPETQRQGASGAVLPLLLPASLAQASLGFWR
WWLHGPDLWSASHHFLQCLSLRKLPLPLFDKNTCGCI
NP_001368870.1
(SEQ ID NO: 4)
MVWRLVLLALWVWPSTQAGHQDKDTTFDLFSISNINRKTIGAKQFRGPDP
GVPAYRFVRFDYIPPVNADDLSKITKIMRQKEGFFLTAQLKQDGKSRGTL
LALEGPGLSQRQFEIVSNGPADTLDLTYWIDGTRHVVSLEDVGLADSQWK
NVTVQVAGETYSLHVGCDLIDSFALDEPFYEHLQAEKSRMYVAKGSARES
HFRGLLQNVHLVFENSVEDILSKKGCQQGQGAEINAISENTETLRLGPHV
TTEYVGPSSERRPEVCERSCEELGNMVQELSGLHVLVNQLSENLKRVVGT
HAGAAGVIGHGQLRRWPISAGINIPWLGSASCSM
NP_001368871.1
(SEQ ID NO: 5)
MRQKEGFFLTAQLKQDGKSRGTLLALEGPGLSQRQFEIVSNGPADTLDLT
YWIDGTRHVVSLEDVGLADSQWKNVTVQVAGETYSLHVGCDLIDSFALDE
PFYEHLQAEKSRMYVAKGSARESHFRGLLQNVHLVFENSVEDILSKKGCQ
QGQGAEINAISENTETLRLGPHVITEYVGPSSERRPEVCERSCEELGNMV
QELSGLHVLVNQLSENLKRVSNDNQFLWELIGGPPKTRNMSACWQDGRFF
AENETWVVDSCTTCTCKKFKTICHQITCPPATCASPSFVEGECCPSCLHS
VDGEEGWSPWAEWTQCSVTCGSGTQQRGRSCDVISNTCLGPSIQTRACSL
SKCDTRIRQDGGWSHWSPWSSCSVTCGVGNITRIRLCNSPVPQMGGKNCK
GSGRETKACQGAPCPIDGRWSPWSPWSACTVTCAGGIRERTRVCNSPEPQ
YGGKACVGDVQERQMCNKRSCPVDGCLSNPCFPGAQCSSFPDGSWSCGSC
PVGFLGNGTHCEDLDECALVPDICFSTSKVPRCVNTQPGFHCLPCPPRYR
GNQPVGVGLEAAKTEKQVCEPENPCKDKTHNCHKHAECIYLGHFSDPMYK
CECQTGYAGDGLICGEDSDLDGWPNLNLVCATNATYHCIKDNCPHLPNSG
QEDFDKDGIGDACDDDDDNDGVTDEKDNCQLLENPRQADYDKDEVGDRCD
NCPYVHNPAQIDTDNNGEGDACSVDIDGDDVFNERDNCPYVYNTDQRDTD
GDGVGDHCDNCPLVHNPDQTDVDNDLVGDQCDNNEDIDDDGHQNNQDNCP
YISNANQADHDRDGQGDACDPDDDNDGVPDDRDNCRLVENPDQEDLDGDG
RGDICKDDFDNDNIPDIDDVCPENNAISETDFRNFQMVPLDPKGTTQIDP
NWVIRHQGKELVQTANSDPGIAVGFDEFGSVDFSGTFYVNTDRDDDYAGF
VFGYQSSSRFYVVMWKQVTQTYWEDQPTRAYGYSGVSLKVVNSTIGTGEH
LRNALWHIGNTPGQVRILWHDPRNIGWKDYTAYRWHLTHRPKTGYIRVLV
HEGKQVMADSGPIYDQTYAGGRLGLFVFSQEMVYFSDLKYECRDI

The mRNA sequence for human TSP2 can be found under accession number NM_001381939.1).

In humans, TSP2 is encoded by the gene THBS2 (Gene ID: 7058).

### B. Advanced Liver Fibrosis

Generally, advanced liver fibrosis refers to fibrosis of the liver characterized as F3 grade fibrosis, or higher, as determined by non-invasive detection using vibration controlled transient elastography. The grade F3 or higher grade fibrosis is graded by LS cut-offs: F3 9.6-11.4 kPa and F4≥11.5 kPa (M Probe); F3 9.3-10.9 kPa and F4≥11.0 kPa (XL Probe) (Kwok et al., Gut; 65:1359-1368 (2016)).

The advanced liver fibrosis may occur as a pathological condition during NAFLD. NAFLD is associated with metabolic derangement and other systemic morbidities. NAFLD has been recognized as an independent risk factor for metabolic syndrome, type 2 diabetes mellitus, cardiovascular disease (CVD), and chronic kidney disease (CKD). The severity of NAFLD is associated with disease manifestations.

NAFLD includes a wide spectrum of liver conditions ranging from simple steatosis to nonalcoholic steatohepatitis (NASH) and advanced hepatic fibrosis. Therefore, advanced liver fibrosis may or may not be present in NASH.

Steatosis, also called fatty change, is abnormal retention of fat (lipids) within a cell or organ. Steatosis can be present in the liver—the primary organ of lipid metabolism— where the condition is commonly referred to as fatty liver disease.

Vibration controlled transient elastography (VCTE™), provided by Fibroscan® (Echosens, Paris, France), is one of the non-invasive tests for detecting liver steatosis and liver fibrosis using controlled attenuation parameter (CAP™) and liver stiffness (LS), respectively.

Fibroscan® measurements typically include different probes for measurement accuracy and consistency. The range of probe models typically match the measurement area of most patient morphology. By adjusting the measurement area relative to the distance of the liver below the surface of the skin, a consistent three cubic centimeters explored volume can be maintained. This is achieved through three probes:
S+ Probe, Pediatric: Designed for pediatric patients with a thoracic perimeter less than 75 cm;
M+ Probe, Medium: Designed for adults in which the distance from the skin to the liver capsule is 25 mm or less; and
XL+ Probe, Extra Large: Designed for heavier weight adults in which the distance from the skin to the liver capsule is over 35 mm.

Typically, non-invasive liver fibrosis (stiffness) may be measured by VCTE™, and non-invasive liver steatosis may be measured by CAP™ Stiffness (kPa) and CAP (dB/m) measurements can be measured at the same time using FibroScan@. The scan's S, M, and XL probes are compatible with all morphologies of patients. CAP is a tool for non invasive assessment and quantification of steatosis. CAP is a measure of the ultrasound attenuation which corresponds to the decrease in amplitude of ultrasound waves as they propagate through the liver. CAP is powered by a sophisticated guidance process based on VCTE that ensures that: CAP and liver stiffness are simultaneously measured in the same liver volume; CAP is expressed in decibel per meter (dB/m).

Hepatic steatosis may be graded by published CAP cut-offs: Mild steatosis 248-267 dB/m, moderate steatosis 268-279 dB/m and severe steatosis ≥280 dB/m (Karlas et al., J Hepatol; 66:1022-1030 (2017)).

Advanced liver fibrosis may be detected in the presence or absence of mild, moderate, or severe hepatic steatosis. Table 1 demonstrates that advanced liver fibrosis was detected in subjects having mild, moderate, or severe hepatic steatosis.

### C. Circulating TSP2 as Biomarker for Advanced Liver Fibrosis

The circulating levels of TSP2 in a subject may be used for detecting the presence of advance liver fibrosis or for detecting risk of developing advanced liver fibrosis in the subject.

### 1. TSP2 for Detecting Advanced Liver Fibrosis

Circulating levels of TSP2 show significant association with the presence of advanced liver fibrosis (≥F3 fibrosis) at first assessment.

The circulating levels of TSP2 are typically informative of the presence or risk of developing advanced liver fibrosis when circulating levels of TSP2 are about or over 2 ng/ml. Typically, the circulating levels of TSP2 are measured with assays that detect TSP2 at a nanogram/ml scale. The circulating levels of TSP2 in a sample taken from a subject may range between about 0.2 ng/ml to about 10 ng/ml.

The circulating levels of TSP2 typically are biomarkers of existing advanced liver fibrosis and detect advanced liver fibrosis when the circulating levels of TSP2 in the sample from the subject are greater than about 3.6 ng/ml, such as between about 3.6 ng/ml and 10 ng/ml. Typically, the circulating levels of TSP2 can detect advanced liver fibrosis with a sensitivity of about or greater than 80%, specificity of about or greater than 60%, and negative predictive value of about or over 90% when the circulating levels of TSP2 in the sample from the subject are greater than about 3.6 ng/ml.

When combined with other physiologic and/or radiographic biomarkers, the detection sensitivity may be about or greater than 80% and the specificity may be about or greater than 80%. The circulating levels of TSP2 can detect advanced liver fibrosis with a sensitivity of about or greater than 80% and specificity of about or greater than 80%, when the circulating levels of TSP2 when combined with body mass index (BMI) and serum aspartate aminotransferase (AST).

In subjects with type 2 diabetes, the circulating levels of TSP2 of about 3.6 ng/ml may be a cut-off value for detecting advanced liver fibrosis. Subjects with type 2 diabetes may have advanced liver fibrosis when the circulating levels of TSP2 are about or greater than 3.6 ng/ml. 2. TSP2 for Risk of Developing Advanced Liver Fibrosis Circulating levels of TSP2 at first assessment also show significant association with liver fibrosis progression and can be used to detect risk of developing advanced liver fibrosis (≥F3 fibrosis) over time.

The circulating levels of TSP2 are typically informative for predicting the risk of developing advanced liver fibrosis. Typically, the circulating levels of TSP2 are measured with methods that detect TSP2 at a nanogram/ml scale.

Typically, detecting the risk of developing advanced liver fibrosis over time includes measuring the circulating levels of the TSP2 biomarker and detecting the risk of developing advanced liver fibrosis. Typically, a subject has a 2.82 times-greater risk of developing of advanced liver fibrosis over time per unit increase in the log-transformed serum TSP2 level measured in ng/ml. Typically, the period of time is between about 0.1 years and about 3 years from obtaining the sample from the subject, from measuring the circulating levels of TSP2 in the sample, or both.

When combined with other physiologic and/or radiographic biomarkers, the detection of the risk of developing advanced liver fibrosis has greater accuracy. For example, the circulating levels of TSP2 can detect the risk of developing advanced liver fibrosis with greater NRI and IDI when the circulating levels of TSP2 are combined with body mass index (BMI), platelet count and CAP values.

In subjects who may have one or more of the diseases metabolic syndrome, type 2 diabetes mellitus, cardiovascular disease (CVD), and chronic kidney disease (CKD), the method may detect the risk of developing advanced liver fibrosis over time in patients.

### 3. Subject

The subjects benefiting from the disclosed methods are humans. The subject may be healthy, suffering from, or susceptible to a disease, disorder or condition.

The subject may be free of disease. The subject may have one or more of diseases metabolic syndrome, type 2 diabetes mellitus, CVD, and CKD. The subject may have metabolic syndrome with one or more of obesity, insulin resistance, diabetes mellitus, dyslipidemia, and hypertension. The subject may have diabetes, liver disease, or a combination of diabetes and liver disease. The subject may have type 2 diabetes. The subject may have NAFLD. The subject may have type 2 diabetes and NAFLD. The subject may or may not have NASH.

### III. Methods of Measuring TSP2

### A. Assays for Measuring

### 1. Affinity Chromatography

The circulating levels of TSP2 may be detected by affinity chromatography using resins or columns with immobilized TSP2 ligands or antibodies to TSP2. The target protein TSP2 is typically adsorbed from the sample or diluted sample as it passes through the column while the other substances are washed away. The target is then eluted and made available for analysis by reversing the prevailing experimental conditions.

TSP2 binds to extracellular matrix ligands including, TGF-beta-1, histidine rich glycoprotein, TSG6, heparin, MMP-2, and heparan sulfate proteoglycans. TSP2 binds to cell surface receptors including CD36, CD47, LDL receptor-related protein-1 (via calreticulin) and the integrins alpha-V/beta-3, alpha-4/beta-1, and alpha-6/beta-1. The chromatography columns may also include antibodies or antibody binding fragments for capturing TSP2. Any one or more of these ligands may be a capture reagent, or a set of capture reagents, used in affinity chromatography for capture and purification of TSP2.

Once separated and eluted from the column, the concentration of TSP2 can be detected using standard protein quantification assays.

### 2. Immunoassays

The methods for measuring circulating levels of TSP2 include an immunoassay whereby polypeptides of the biomarker are evaluated or detected by their interaction with a biomarker-specific antibody, antibody binding fragment, a combination of different antibodies, or a combination of different antibody binding fragments. The biomarker can be detected in either a qualitative or quantitative manner. Exemplary immunoassays that can be used for the detection of the biomarker polypeptides and proteins include, but are not limited to, radioimmunoassays, ELISAs, immunoprecipitation assays, Western blot, fluorescent immunoassays, and immunohistochemistry, flow cytometry, protein arrays, multiplexed bead arrays, magnetic capture, in vivo imaging, fluorescence resonance energy transfer (FRET), and fluorescence recovery/localization after photobleaching (FRAP/FLAP).

Some immunoassays, for example ELISAs, can require two different biomarker specific antibodies or ligands (e.g., a capture ligand or antibody, and a detection ligand or antibody). In certain embodiments, the protein biomarker is captured with a ligand or antibody on a surface and the protein biomarker is labeled with an enzyme. In one example, a detection antibody conjugated to biotin or streptavidin can be used to create a biotin-streptavidin linkage to an enzyme that contains biotin or streptavidin. A signal is generated by the conversion of the enzyme substrate into a colored molecule and the intensity of the color of the solution is quantified by measuring the absorbance with a light sensor. Assays may utilize chromogenic reporters and substrates that produce an observable color change to indicate the presence of the protein biomarker. Fluorogenic, electrochemiluminescent, and real-time PCR reporters are also contemplated to create quantifiable signals.

Some assays optionally including fixing one or more antibodies to a solid support to facilitate washing and subsequent isolation of the complex, prior to contacting the antibody with a sample. Examples of solid supports include glass or plastic in the form of, e.g., a microtiter plate, a stick, a bead, or a microbead. Antibodies can also be attached to a probe, substrate or a PROTEINCHIP® array.

Flow cytometry is a laser based technique that may be employed in counting, sorting, and detecting protein biomarkers by suspending particles in a stream of fluid and passing them by an electronic detection apparatus. A flow cytometer has the ability to discriminate different particles on the basis of color. Differential dyeing of particles with different dyes, emitting in two or more different wavelengths allows the particle to be distinguished. Multiplexed analysis, such as FLOWMETRIX™ is discussed in Fulton, et al., Clinical Chemistry, 43(9):1749-1756 (1997) and can allow one to perform multiple discrete assays in a single tube with the same sample at the same time.

In some specific embodiments, the biomarker level(s) are measured using LUMINEX XMAP® technology. LUMINEX XMAP® is frequently compared to the traditional ELISA technique, which is limited by its ability to measure only a single analyte. The differences between ELISA and LUMINEX XMAP® technology center mainly on the capture antibody support. Unlike with traditional ELISA, LUMINEX XMAP® capture antibodies are covalently attached to a bead surface, effectively allowing for a greater surface area as well as a matrix or free solution/liquid environment to react with the analytes. The suspended beads allow for assay flexibility in a singleplex or multiplex format.

Commercially available formats that include Luminex xMAP® technology includes, for example, BIO-PLEX® multiplex immunoassay system which permits the multiplexing of up to 100 different assays within a single sample. This technique involves 100 distinctly colored bead sets created by the use of two fluorescent dyes at distinct ratios. These beads can be further conjugated with a reagent specific to a particular bioassay. The reagents may include antigens, antibodies, oligonucleotides, enzyme substrates, or receptors. The technology enables multiplex immunoassays in which one antibody to a specific analyte is attached to a set of beads with the same color, and the second antibody to the analyte is attached to a fluorescent reporter dye label. The use of different colored beads enables the simultaneous multiplex detection of many other analytes in the same sample. A dual detection flow cytometer can be used to sort out the different assays by bead colors in one channel and determine the analyte concentration by measuring the reporter dye fluorescence in another channel.

In some specific embodiments, the biomarker(s) levels are measured using Quanterix's SIMOA™ technology. SIMOA™ technology (named for single molecule array) is based upon the isolation of individual immunocomplexes on paramagnetic beads using standard ELISA reagents. The main difference between Simoa and conventional immunoassays lies in the ability to trap single molecules in femtoliter-sized wells, allowing for a “digital” readout of each individual bead to determine if it is bound to the target analyte or not. The digital nature of the technique allows an average of 1000× sensitivity increase over conventional assays with CVs <10%. Commercially available SIMOA™ technology platforms offer multiplexing options up to a 10-plex on a variety of analyte panels, and assays can be automated.

Multiplexing experiments can generate large amounts of data. Therefore, in some embodiments, a computer system is utilized to automate and control data collection settings, organization, and interpretation.

Typically, the assays for detecting TSP2 include immunoassays at a lowest detection limit between about 0.01 ng/ml and about 0.18 ng/ml, such as between about 0.01 ng/ml and about 0.16 ng/ml, or about 0.156 ng/ml. In some embodiments, the assays for detecting TSP2 have intra- and inter-assay precision of less than 4.6% and less than 7.2%, respectively.

### 3. Controls

The methods including analyzing one or more biomarkers typically includes comparing to a control. For example, the level of a biomarker detected in a sample obtained from the subject can be compared to a control.

Suitable controls will be known to one of skill in the art. Controls can include, for example, standards obtained from healthy subjects, such as subjects without the disease or disorder, or non-diseased tissue from the same subject. A control can be a single or pooled or averaged values of like individuals using the same assay. Reference indices can be established by using subjects that have been diagnosed with the disease or disorder with different known disease severities or prognoses.

### B. Sensitivity and Specificity of Detecting Liver Fibrosis

The sensitivity can be expressed as a percentage, the proportion of actual positives which are correctly identified as such (e.g., the percentage of test subjects having advanced liver fibrosis correctly identified by the test as having advanced liver fibrosis). A test with high sensitivity has a low rate of false negatives, i.e., the cases of advanced liver fibrosis not identified as such. Generally, the disclosed assays and methods have a sensitivity of at least 80%, of at least 90%, at least 92%, at least 94%, at least 95%, at least 96%, at least 97%, at least 98%, at least 99%, or at least 100%.

The specificity can be expressed as a percentage, the proportion of actual negatives which are correctly identified as such (e.g., the percentage of test subjects not having advanced liver fibrosis correctly identified by the test as not having advanced liver fibrosis). A test with high specificity has a low rate of false positives, i.e., the cases of individuals not having advanced liver fibrosis but suggested by the test as having advanced liver fibrosis. Generally, the disclosed methods have a specificity of at least 60%, at least 70%, at least 80%, at least 90%, at least 92%, at least 94%, at least 95%, at least 96%, at least 97%, at least 98%, at least 99%, or at least 100%.

Positive and negative predictive values are influenced by the prevalence of disease in the population that is being tested. In a high prevalence setting, it is more likely that persons who test positive truly have disease than if the test is performed in a population with low prevalence.

Typically, the method detects advanced liver fibrosis with a sensitivity of about or greater than 80%, specificity of about or greater than 60%, and negative predictive value of about or over 90% when the circulating levels of TSP2 in the sample from the subject are greater than about 3.6 ng/ml.

### IV. Kits for Measuring TSP2

A set of capture reagents having a binding specificity to TSP2 may be packaged together in any suitable combination as a kit useful for performing, or aiding in the performance of, the disclosed method. It is useful if the kit components in a given kit are designed and adapted for use together in the disclosed method.

For example, disclosed are kits with one or more sets of capture reagents. The kits may include dilution buffers, sample buffers, control reagents, elution reagents, and detection reagents. The kits may include a substrate for capture, elution, and detection of TSP2. The kits may include a substrate for capture and detection of TSP2. The kits may include instructions use.

### EXAMPLES

### Example 1. Serum TSP2 Level as a Diagnostic and Prognostic Marker for Advanced Fibrosis in Non-Alcoholic Fatty Liver Disease with Type 2 Diabetes

### Research Design & Methods

### Study Participants

All participants were recruited from the Hong Kong West Diabetes NAFLD Cohort, which consisted of patients who were regularly followed up at the diabetes clinic of Queen Mary Hospital, Hong Kong. Consecutive patients who were Chinese, aged between 21 and 80 years, and attended diabetes complications screening since January 2017, were invited to undergo a prospective study that aimed to identify the risk factors of NAFLD fibrosis progression in type 2 diabetes. VCTE was used for the assessment of their hepatic steatosis and fibrosis at regular intervals. Patients who had active malignancy, concomitant chronic hepatitis B or C, or documented history of any other liver disease including alpha-1 anti-trypsin deficiency, Wilson's disease, autoimmune hepatitis, drug-induced liver injury, primary biliary cholangitis, or chronic use of steatogenic medications such as amiodarone, methotrexate or tamoxifen, were excluded. Furthermore, patients with daily alcohol consumption of more than 30 g in men or 20 g in women were also excluded (Chalasani et al., Hepatology; 67:328-357 (2018)). The study protocol was approved by the institutional review board of the University of Hong Kong/Hospital Authority Hong Kong West Cluster. Written informed consent was obtained from all recruited participants prior to any study-related procedures.

In the current study, which evaluated the relationship between circulating TSP2 level and NAFLD in type 2 diabetes, only participants who had hepatic steatosis at baseline and recruited between January 2017 and June 2020 were included. Moreover, in the analysis examining the prospective association of circulating TSP2 level with hepatic fibrosis progression, only participants who did not have advanced fibrosis or cirrhosis at baseline were included. The levels of hepatic steatosis and fibrosis were defined by controlled attenuation parameter (CAP) and liver stiffness (LS) measurements on vibration controlled transient elastography (VCTE), respectively.

### Clinical and Biochemical Assessments

All patients from the diabetes clinic had regular complications assessment as part of the standard clinical management. This is to ascertain their glycemic control, cardiovascular risk factors and the presence of diabetic complications. Anthropometric parameters, including body weight (BW), height (BH), body mass index (BMI), waist circumference (WC), and blood pressure (BP) were measured. Fasting blood was drawn for plasma glucose, lipids, glycated hemoglobin (HbA1c), complete blood count, liver and renal function tests. Albuminuria status was assessed with a random urine sample, and categorized according to their urine albumin to creatinine ratio (<30 mg/g [A1], ≥30-<300 mg/g [A2] and >300 mg/g [A3]). Moreover, all patients received regular retinal photographs and/or assessments by ophthalmologists. For those who consented to participate in the NAFLD cohort study, smoking status, alcohol consumption, detailed medical, drug and family histories were obtained using a standardized questionnaire, and prothrombin time was also checked. Moreover, fasting blood was stored in aliquots at −70° C. for assays of emerging NAFLD biomarkers.

Conventional fibrosis scores including NAFLD fibrosis score (NFS) and Fibrosis-4 index (FIB-4) were determined using published formula and categorized based on recommended cut-offs (Vilar-Gomez and Chalasani. J Hepatol; 68:305-315 (2018)).

### Measurement of Serum TSP2 Levels

Serum TSP2 levels were measured with an enzyme-linked immunosorbent assay (ELISA) kit for human TSP-2, using a pair of monoclonal antibodies which recognized distinct sites of human TSP2 (Antibody and Immunoassay Services, University of Hong Kong). The antibodies used were IgGs. Serum samples were 2-fold diluted in this assay. Secondary antibody was biotin labelled.

The assay was highly specific to human TSP2 and did not show any cross-reactivity to human TSP1, TSP3, TSP4 and TSP5. The lowest detection limit was 0.156 ng/ml, with its intra- and inter-assay precision being <4.6% and <7.2%, respectively.

### Vibration Controlled Transient Elastography

All participants received VCTE at baseline, and every 12-18 months thereafter for reassessment. VCTE was performed after fasting for at least 8 hours. CAP and LS were measured using Fibroscan@ (Echosens, Paris, France), by two operators with experience in performing over 500 measurements. The inter-observer reliability was satisfactory, as reflected by an intra-class correlation of 0.98 for CAP and 0.97 for LS measurements. Both CAP and LS were represented by the median of 10 reliable measurements, defined when the inter-quartile range was less than 30% and the success rate was more than 60%. Only CAP values with interquartile range of 40 dB/m or above were used to ensure validity of the results (Wong et al., J Hepatol; 67:577-584 (2017)). All examinations were done using M probe in the first attempt, and XL probe was used if the BMI was more than 30 kg/m².

Hepatic steatosis was graded by published CAP cut-offs: Mild steatosis 248-267 dB/m, moderate steatosis 268-279 dB/m and severe steatosis ≥280 dB/m (Karlas et al., J Hepatol; 66:1022-1030 (2017)) Advanced fibrosis (F3) and cirrhosis (F4) were graded by LS cut-offs: F3 9.6-11.4 kPa and F4≥11.5 kPa (M Probe); F3 9.3-10.9 kPa and F4≥11.0 kPa (XL Probe) (Kwok et al., Gut; 65:1359-1368 (2016)). Fibrosis progression was defined as the development of ≥F3 fibrosis (i.e. advanced fibrosis or cirrhosis) upon reassessment VCTE as of 31 Dec. 2020.

### Definitions of Clinical Variables

Central obesity was defined as WC≥80 cm in women and 90 cm in men. Hypertension was defined as BP≥140/90 mmHg or on anti-hypertensive medications. Dyslipidemia was defined as fasting triglycerides (TG)≥150 mg/dL, high density lipoprotein cholesterol (HDL-C)<40 mg/dL in men and <50 mg/dL in women, and low density lipoprotein cholesterol (LDL-C)≥100 mg/dL, or on lipid-lowering agents. The diagnoses of coronary heart disease (CHD) and stroke were based on diagnostic codes from the 9^{th} edition of the International Classification of Diseases (ICD-9) (410, 36.01-10 for CHD and 430-438 for stroke).

### Statistical Analysis

All data were analysed with R version 3.6.0 (MatchIt Package, DeLong's test for two correlated receiver operating characteristics curves) and IBM SPSS Statistics version 26.0 (IBM Corp., Armonk, New York). Data that were not normally distributed as determined by Kolmogorov-Smirnov test, such as serum TG, ALT, AST, FIB4 and TSP2 levels, were logarithmically transformed to obtain near normality before analysis. Values were reported as means±standard deviation (SD), medians with 25^{th} and 75^{th} percentiles (for variables if skewed data), or percentages, as appropriate. Chi-square test and ANOVA were used for comparisons of categorical and continuous variables, respectively. Multivariable logistic regression analysis was performed to evaluate the independent determinants of the presence and development of ≥F3 fibrosis, based on models with the lowest Akaike Information Criteria (AIC). Variables that were statistically significant in univariate analysis were included in the multivariable logistic regression analysis. The area under the receiver operating characteristics curves (AUROC) of serum TSP2 with and without the addition of clinical risk factors were determined, while AUROCs of different clinical models were compared using the DeLong method. The optimal cut-off of serum TSP2 level to identify the presence of ≥F3 fibrosis was derived based on the point with maximum Youden j index (y) on ROC curve with y=[sensitivity−(1−specificity)]. The predictive performance of the various models was further evaluated using category-free net reclassification improvement (NRI) and integrated discrimination improvement (IDI). In all statistical tests, a two-sided p-value <0.05 was considered significant.

### Results

Serum TSP2 Level was Significantly Associated with the Presence of ≥F3 Fibrosis in Type 2 Diabetes

Among the 820 participants with type 2 diabetes and NAFLD included in this study, 138 (16.8%) of them had ≥F3 fibrosis at baseline. Table 1 summarizes the baseline characteristics of the study participants. Participants who had ≥F3 fibrosis were significantly younger, with higher BMI, WC, serum TG, ALT, AST, and lower HDL-C levels and platelet count than those who did not. Moreover, their duration of diabetes was significantly shorter with a higher prevalence of albuminuria than those without ≥F3 fibrosis. Furthermore, participants with ≥F3 fibrosis had significantly higher CAP, NFS and FIB4 values than those who did not (CAP: 339 dB/m vs. 299 dB/m, p<0.001; NFS: −0.75 vs. −1.13, p=0.001; FIB4: 1.26 vs 1.05, p<0.001, respectively). Of note, median serum TSP2 levels were significantly higher in participants with ≥F3 fibrosis than those without (4.17 ng/ml vs. 2.33 ng/ml, respectively; p<0.001).

TABLE 1
Baseline characteristics of the study participants stratified
by presence of ≥F3 fibrosis (N = 820)

| | Without ≥F3 | With ≥F3 | p- |
|---|---|---|---|
| Variable | fibrosis | fibrosis | value |
| N | 682 | 138 | |
| Clinical | | | |
| variables | | | |
| Men, % | 51.2 | 52.2 | 0.806 |
| Age, years | 59.9 ± 10.2 | 57.8 ± 11.3 | 0.037 |
| Ever smoker, % | 24.5 | 29.0 | 0.267 |
| Duration of | 17.1 ± 9.5 | 15.1 ± 7.9 | 0.022 |
| diabetes, years | | | |
| BMI, kg/m² | 27.7 ± 4.1 | 31.4 ± 5.2 | <0.001 |
| WC, cm | 95.3 ± 10.3 | 105.0 ± 15.0 | <0.001 |
| Central obesity, % | 85.6 | 95.7 | 0.001 |
| Systolic BP, mmHg | 136.0 ± 16.6 | 137.0 ± 15.5 | 0.553 |
| Diastolic BP, mmHg | 77.8 ± 9.7 | 78.9 ± 9.2 | 0.220 |
| Medical | | | |
| history | | | |
| Hypertension, % | 80.9 | 81.2 | 0.952 |
| Dyslipidemia, % | 95.0 | 94.9 | 0.966 |
| Coronary heart | 18.2 | 16.7 | 0.643 |
| disease, % | | | |
| Stroke, % | 2.2 | 2.2 | 0.985 |
| Use of NAFLD- | | | |
| related medications | | | |
| GLP1rA, % | 0.9 | 2.9 | 0.053 |
| Pioglitazone, % | 13.7 | 17.1 | 0.286 |
| SGLT2i, % | 18.2 | 16.4 | 0.612 |
| Biochemical | | | |
| variables | | | |
| HbA1c, % | 7.63 ± 1.20 | 7.72 ± 1.21 | 0.440 |
| HbA1c, mmol/mol | 59.9 ± 13.1 | 61.0 ± 13.1 | 0.440 |
| HDL-C, mg/dL | 45.7 ± 11.4 | 41.6 ± 11.0 | <0.001 |
| LDL-C, mg/dL | 81.5 ± 28.1 | 79.3 ± 26.8 | 0.404 |
| TG*, mg/dL | 124 | 151 | <0.001 |
| | (92-175) | (110-211) | |
| ALT*, U/L | 22.0 | 38.5 | <0.001 |
| | (17.0-31.0) | (28.0-55.5) | |
| AST*, U/L | 21.0 | 32.0 | <0.001 |
| | (18.0-25.0) | (25.0-42.5) | |
| eGFR, ml/min/1.73 m² | 82.0 ± 25.6 | 80.5 ± 26.6 | 0.536 |
| Albumin, g/L | 44.7 ± 3.2 | 45.0 ± 3.2 | 0.407 |
| Platelets, ×10\hat{ }9/L | 265.0 ± 66.8 | 247.0 ± 68.1 | 0.004 |
| Transient | | | |
| elastography | | | |
| CAP, dB/m | 302.0 ± 41.1 | 338.0 ± 43.0 | <0.001 |
| Steatosis grade | | | <0.001 |
| Mild, % | 24.0 | 6.5 | |
| Moderate, % | 11.6 | 5.1 | |
| Severe, % | 64.4 | 88.4 | |
| Diabetes | | | |
| microvascular | | | |
| complications | | | |
| Diabetic | 54.0 | 53.6 | 0.942 |
| retinopathy, % | | | |
| Albuminuria, % | 47.1 | 61.6 | 0.002 |
| Conventional | | | |
| Fibrosis | | | |
| Scores | | | |
| NFS | −1.13 ± 1.21 | −0.75 ± 1.21 | 0.001 |
| FIB4* | 1.05 | 1.26 | <0.001 |
| | (0.79-1.38) | (0.88-1.69) | |
| Biomarker | | | |
| TSP2*, ng/ml | 2.33 | 4.17 | <0.001 |
| | (1.65-3.21) | (2.75-6.49) | |

*Log-transformed before analysis.

Data were presented as mean±standard deviation or median (25th to 75th percentile).

TSP2, thrombospondin 2; BMI, body mass index; WC waist circumference; BP, blood pressure; NAFLD, non-alcoholic fatty liver disease; GLP1rA, glucagon-like peptide 1 receptor agonists; SGLT2i, sodium-glucose co-transporter 2 inhibitors; HbA1c, glycated hemoglobin; HDL-C, high-density lipoprotein cholesterol; LDL-C, low-density lipoprotein cholesterol; TG, triglycerides; ALT, alanine aminotransferase; AST, aspartate transaminase; eGFR, estimated glomerular filtration rate; CAP, controlled attenuation parameter; NFS, Non-alcoholic fatty liver disease fibrosis score; FIB4, Fibrosis-4 index.

Albuminuria was defined as urine albumin to creatinine ratio ≥30 mg/g; Conversion factors for HDL/LDL-C from mmol/l to mg/dL×38.9; TG from mmol/l to mg/dL×88.2.

The associations of baseline clinical variables with increasing quartiles of serum TSP2 levels of the study participants are summarized in Table 2. Higher quartiles of baseline TSP2 levels were significantly associated with higher BMI (p<0.001), WC (p<0.001), systolic BP (p=0.01), serum HbA1c (p=0.005), TG (p=0.001), ALT (p<0.001), AST levels (p<0.001) and the prevalence of albuminuria (p<0.001), but with lower HDL-C (p=0.02), eGFR (p=0.001), albumin levels (p<0.001) and platelet count (p=0.023) at baseline. Moreover, higher quartiles of baseline TSP2 levels were also significantly associated with higher stages of hepatic steatosis (p<0.001 for CAP) and fibrosis (NFS, FIB4, and LS; all p<0.001).

TABLE 2
Associations of baseline variables with increasing quartiles
of serum TSP2 levels in all study participants (N = 820)

| | TSP ng/ml | | | | |
|---|---|---|---|---|---|
| Variable | Q1: <1.77 | Q2: 1.77-2.46 | Q3: 2.47-3.56 | Q4: >3.56 | p for trend |
| N | 205 | 205 | 205 | 205 | |
| Clinical | | | | | |
| variables | | | | | |
| Men, % | 54.6 | 52.7 | 50.2 | 47.3 | 0.118 |
| Age, years | 58.2 ± 10.7 | 60.1 ± 9.5 | 60.0 ± 9.9 | 59.9 ± 11.4 | 0.112 |
| Ever-smoker, % | 28.3 | 25.9 | 25.4 | 21.5 | 0.451 |
| Duration of | 16.2 ± 9.9 | 16.8 ± 9.2 | 17.6 ± 9.4 | 16.4 ± 8.6 | 0.640 |
| diabetes, years | | | | | |
| BMI, kg/m² | 27.6 ± 3.8 | 27.7 ± 4.2 | 28.1 ± 4.9 | 29.7 ± 4.9 | <0.001 |
| WC, cm | 95.7 ± 9.9 | 95.1 ± 10.8 | 97.0 ± 13.9 | 99.9 ± 11.7 | <0.001 |
| Central obesity, % | 88.3 | 82.9 | 82.9 | 95.1 | <0.001 |
| Systolic BP, mmHg | 134.0 ± 17.5 | 134.0 ± 16.6 | 138.0 ± 16.1 | 137.0 ± 15.1 | 0.010 |
| Diastolic BP, mmHg | 77.9 ± 8.8 | 77.9 ± 10.0 | 78.5 ± 10.3 | 77.6 ± 9.4 | 0.899 |
| Medical | | | | | |
| history | | | | | |
| Hypertension, % | 81.0 | 78.0 | 83.4 | 81.5 | 0.578 |
| Dyslipidemia, % | 94.6 | 94.6 | 95.6 | 95.1 | 0.720 |
| Coronary heart | 17.6 | 15.1 | 22.4 | 17.1 | 0.626 |
| disease, % | | | | | |
| Stroke, % | 2.4 | 2.4 | 2.0 | 2.0 | 0.670 |
| Biochemical | | | | | |
| variables | | | | | |
| HbA1c, % | 7.50 ± 1.23 | 7.53 ± 0.99 | 7.79 ± 1.26 | 7.77 ± 1.28 | 0.005 |
| HbA1c, mmol/mol | 58.5 ± 13.5 | 58.8 ± 10.8 | 61.7 ± 13.8 | 61.4 ± 14.0 | 0.005 |
| HDL-C, mg/dL | 46.0 ± 11.6 | 45.4 ± 10.6 | 45.4 ± 11.2 | 43.2 ± 12.2 | 0.020 |
| LDL-C, mg/dL | 81.8 ± 29.0 | 82.4 ± 26.1 | 83.5 ± 28.6 | 76.8 ± 27.7 | 0.117 |
| TG*, mg/dL | 128 | 121 | 120 | 146 | 0.001 |
| | (91-173) | (94-183) | (90-167) | (103-208) | |
| ALT*, U/L | 19.0 | 22.0 | 23.0 | 33.0 | <0.001 |
| | (16.0-27.0) | (16.0-29.0) | (18.0-35.5) | (23.0-52.0) | |
| AST*, U/L | 20.0 | 20.0 | 23.0 | 28.0 | <0.001 |
| | (17.0-23.0) | (17.0-24.0) | (19.0-29.0) | (22.0-39.0) | |
| eGFR, ml/min/1.73 m² | 86.4 ± 23.5 | 81.3 ± 25.8 | 82.0 ± 26.5 | 77.1 ± 26.6 | 0.001 |
| Albumin, g/L | 45.4 ± 2.71 | 44.6 ± 3.07 | 44.8 ± 3.12 | 44.2 ± 3.74 | <0.001 |
| Platelets, ×10\hat{ }9/L | 265.0 ± 62.6 | 270.0 ± 72.4 | 264.0 ± 64.6 | 251.0 ± 68.2 | 0.023 |
| Transient | | | | | |
| elastography | | | | | |
| CAP, dB/m | 298.0 ± 37.8 | 299.0 ± 42.9 | 312.0 ± 43.4 | 324.0 ± 45.5 | <0.001 |
| Steatosis | | | | | 0.001 |
| grade | | | | | |
| Mild, % | 24.4 | 28.3 | 19.5 | 12.2 | |
| Moderate, % | 11.2 | 13.2 | 10.2 | 7.3 | |
| Severe, % | 64.4 | 58.5 | 70.2 | 80.5 | |
| LS, kPa | 5.75 ± 2.10 | 6.37 ± 4.56 | 6.95 ± 2.94 | 10.60 ± 7.12 | <0.001 |
| Diabetes | | | | | |
| microvascular | | | | | |
| complications | | | | | |
| Diabetic | 53.2 | 49.3 | 58.0 | 55.1 | 0.348 |
| retinopathy, % | | | | | |
| Albuminuria, % | 37.6 | 48.3 | 48.5 | 63.9 | <0.001 |
| Conventional | | | | | |
| fibrosis | | | | | |
| scores | | | | | |
| NFS | −1.20 ± 1.18 | −1.19 ± 1.29 | −1.08 ± 1.13 | −0.80 ± 1.23 | <0.001 |
| FIB4* | 0.97 | 1.04 | 1.09 | 1.26 | <0.001 |
| | (0.73-1.27) | (0.76-1.31) | (0.82-1.43) | (0.90-1.68) | |

TSP2, thrombospondin 2; BMI, body mass index; WC waist circumference; BP, blood pressure; HbA1c, glycated hemoglobin; HDL-C, high-density lipoprotein cholesterol; LDL-C, low-density lipoprotein cholesterol; TG, triglycerides; ALT, alanine aminotransferase; AST, aspartate transaminase; eGFR, estimated glomerular filtration rate; CAP, controlled attenuation parameter; LS, liver stiffness; NFS, non-alcoholic fatty liver disease fibrosis score; FI1B4, fibrosis-4 index. Albuminuria was defined as urine albumin to creatinine ratio ≥30 mg/g; Conversion factors for HDL/LDL-C from mmol/l to mg/dL×38.9; TG from mmol/l to mg/dL×88.2. Multivariable logistic regression analysis was performed, which included age, BMI, duration of diabetes, platelet count, serum HDL-C, TG, ALT, AST, CAP, TSP2 levels and albuminuria. Serum TSP2 level was independently associated with the presence of ≥F3 fibrosis at baseline (Odds ratio OR 5.13, 95% CI 3.16-8.32, p<0.001), together with BMI (OR 1.14, 95% CI 1.08-1.20, p<0.001), serum AST (OR 8.01, 95% CI 4.10-15.60, p<0.001) and CAP values (OR 1.008, 95% CI 1.002-1.014, p=0.01) (Table 3).

TABLE 3
Multivariable logistic regression analysis showing the
association of serum TSP2 level with presence of ≥F3
fibrosis at baseline (N = 820)

Model

| Baseline variable | OR (95% CI) | p-value |
|---|---|---|
| BMI, kg/m² | 1.14 | <0.001 |
| | (1.08-1.20) | |
| AST*, U/L | 8.01 | <0.001 |
| | (4.10-15.6) | |
| CAP, dB/m | 1.008 | 0.010 |
| | (1.002-1.014) | |
| TSP2*, ng/ml | 5.13 | <0.001 |
| | (3.16-8.32) | |

*Log-transformed before analysis.

Variables included in the analysis consisted of age, BMI, duration of diabetes, albuminuria, HDL-C, TG, ALT, AST, platelet count, CAP and TSP2 levels. Model selection was based on Akaike Information Criteria. TSP2, thrombospondin 2; BMI, body mass index; HDL-C, high-density lipoprotein cholesterol; TG, triglyceride; ALT, alanine aminotransferase; AST, aspartate aminotransferase; CAP, controlled attenuation parameter; OR, odds ratio; 95% CI, 95% confidence interval. Albuminuria was defined as urine albumin to creatinine ratio ≥30 mg/g.

The results were similar if BMI was replaced by WC (OR 5.67, 95% CI 3.49-9.22, p<0.001 for TSP2 and OR 1.06, 95% CI 1.04-1.08, p<0.001 for WC). Moreover, in a subgroup analysis, the association between serum TSP2 level and ≤F3 fibrosis remained significant regardless of the levels of NFS or FIB4, although the associations were apparently stronger in those with higher conventional non-invasive fibrosis scores (Table 4).

TABLE 4
Subgroup analysis showing the associations of serum TSP2 level
with ≥F3 fibrosis in study participants stratified by
their levels of conventional non-invasive fibrosis scores.

| | | | Model | |
|---|---|---|---|---|
| | | | Adjusted OR of TSP2* | |
| | N | ≥F3 fibrosis, % | (95% CI) | p-value |
| FIB-4 | | | | |
| <1.3 | 556 | 13.7 | 3.02 | <0.001 |
| | | | (1.66-5.49) | |
| ≥1.3 | 264 | 23.5 | 14.0 | <0.001 |
| | | | (5.32-36.7) | |
| NFS | | | | |
| <−1.455 | 297 | 14.1 | 2.98 | 0.006 |
| | | | (1.36-6.52) | |
| ≥1.455 | 523 | 18.4 | 7.09 | <0.001 |
| | | | (3.74-13.4) | |

*Log-transformed before analysis.
Model included BMI, AST and CAP.
TSP2, thrombospondin-2; OR, odds ratio; 95% CI, 95% confidence interval; FIB4, Fibrosis-4 index; NFS, non-alcoholic fatty liver disease fibrosis score; BMI, body mass index; AST, aspartate aminotransferase; CAP, controlled attenuation parameter.

### Performance of Serum TSP2 to Identify the Presence of F3 Fibrosis on VCTE

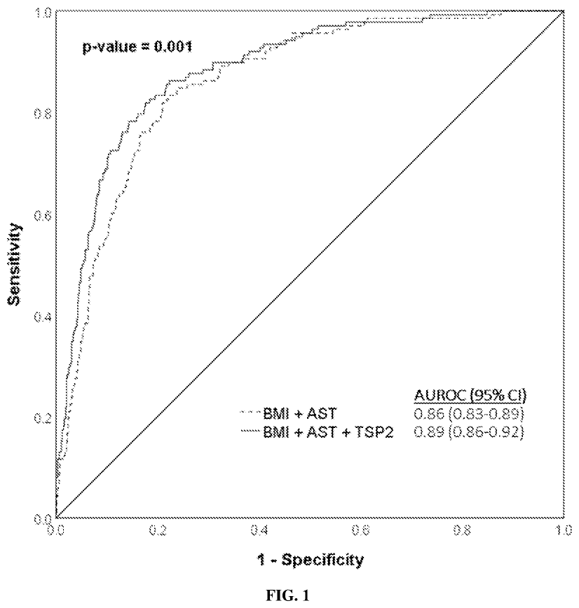

Next, serum TSP2 level was studied for whether it was clinically useful in identifying individuals with ≥F3 fibrosis on VCTE who might need referral to hepatologist for further assessments. The AUROC of serum TSP2 alone to indicate ≥F3 fibrosis on VCTE was 0.80 (95% CI 0.76-0.84). Notably, when serum TSP2 level was added to a clinical model consisting of BMI and serum AST, the two other independent determinants of ≥F3 fibrosis, the AUROC increased significantly from 0.86 (95% CI, 0.83-0.89) to 0.89 (95% CI 0.86-0.92, p=0.01) (FIG. 1). This was accompanied by significant improvements in both the NRI (60.7, 95% CI 53.1-78.3, p<0.001) and IDI (8.1, 95% CI 5.0-11.0, p<0.001). The use of an optimal serum TSP2 cut-off of 3.6 ng/ml to identify the presence of ≥F3 fibrosis yielded a sensitivity of 83.6%, specificity of 64.5%, positive predictive value (PPV) of 44.3% and negative predictive value (NPV) of 92.1%.

Baseline Serum TSP2 Level was Independently Associated with the Development of ≥F3 Fibrosis in Patients with Type 2 Diabetes

Among the 682 participants who did not have ≥F3 fibrosis at baseline, 491 had received reassessment VCTE during the study period, after excluding 90 participants who refused to come back due to coronavirus disease 2019 (COVID-19), 17 who refused further TE, 19 who were lost to follow-up, 6 who had died and 59 who were not due for reassessment TE. Over a median follow-up of 1.5 years, 43 (8.8%) out of 491 participants had developed ≥F3 fibrosis.

Participants with incident ≥F3 fibrosis were significantly younger, had higher baseline BMI, WC, ALT, AST, CAP and NFS levels, and lower serum HDL-C and platelet count than those without. Importantly, participants who developed ≥F3 fibrosis had significantly higher baseline serum TSP2 level than those who did not (3.21 ng/ml vs. 2.29 ng/ml, p<0.001) (Table 5).

TABLE 5
Baseline clinical characteristics of the study participants
stratified by hepatic fibrosis progression (N = 491)

| | Without hepatic | With hepatic | |
|---|---|---|---|
| | fibrosis | fibrosis | |
| Baseline variable | progression | progression | p-value |
| N | 448 | 43 | |
| Clinical | | | |
| variables | | | |
| Men, % | 51.3 | 60.5 | 0.253 |
| Age, years | 59.8 ± 10.1 | 56.6 ± 9.2 | 0.045 |
| Ever smoker, % | 25.2 | 16.3 | 0.192 |
| Duration of | 17.2 ± 9.4 | 15.7 ± 9.2 | 0.298 |
| diabetes, years | | | |
| BMI, kg/m² | 27.4 ± 3.8 | 31.0 ± 5.0 | <0.001 |
| WC, cm | 94.6 ± 10.1 | 104.4 ± 10.6 | <0.001 |
| Systolic BP, mmHg | 136.0 ± 16.7 | 135.0 ± 15.1 | 0.752 |
| Diastolic BP, mmHg | 77.8 ± 9.8 | 78.6 ± 9.8 | 0.621 |
| Medical | | | |
| history | | | |
| Hypertension, % | 80.4 | 81.4 | 0.873 |
| Dyslipidemia, % | 94.0 | 97.7 | 0.317 |
| Coronary heart | 19.2 | 16.3 | 0.641 |
| disease, % | | | |
| Stroke, % | 1.8 | 2.3 | 0.565 |
| NAFLD-related | | | |
| Medications | | | |
| GLP1rA, % | 1.1 | 0.0 | 1.00 |
| Pioglitazone, % | 11.8 | 20.9 | 0.086 |
| SGLT2i, % | 19.9 | 14.0 | 0.348 |
| Biochemical | | | |
| variables | | | |
| HbA1c, % | 7.65 ± 1.19 | 7.79 ± 1.34 | 0.472 |
| HbA1c, mmol/mol | 60.1 ± 13.1 | 61.6 ± 14.6 | 0.472 |
| HDL-C, mg/dL | 45.9 ± 11.5 | 41.3 ± 8.3 | 0.011 |
| LDL-C, mg/dL | 81.5 ± 27.6 | 87.9 ± 26.0 | 0.144 |
| TG*, mg/dL | 122 | 137 | 0.177 |
| | (91-170) | (107-182) | |
| ALT*, U/L | 22 | 30 | 0.001 |
| | (17-31) | (21-42) | |
| AST*, U/L | 21 | 24 | 0.002 |
| | (18-25) | (20-31) | |
| eGFR, ml/min/1.73 m² | 83.1 ± 24.3 | 81.3 ± 26.3 | 0.649 |
| Albumin, g/L | 44.7 ± 3.1 | 44.1 ± 4.2 | 0.175 |
| Platelets, ×10\hat{ }9/L | 269.0 ± 68.4 | 240.0 ± 53.2 | 0.008 |
| Transient | | | |
| elastography | | | |
| CAP | 301 ± 41 | 339 ± 36 | <0.001 |
| Severe steatosis, % | 62.3 | 93.0 | <0.001 |
| Diabetes | | | |
| microvascular | | | |
| complications | | | |
| Diabetic | 52.5 | 48.8 | 0.650 |
| retinopathy, % | | | |
| Albuminuria, % | 46.4 | 51.2 | 0.552 |
| Conventional | | | |
| non-invasive | | | |
| fibrosis | | | |
| scores | | | |
| NFS | −1.22 ± 1.21 | −0.72 ± 0.99 | 0.010 |
| FIB4* | 1.04 | 1.10 | 0.335 |
| | (0.76-1.35) | (0.84-1.52) | |
| Biomarker | | | |
| TSP2*, ng/ml | 2.29 | 3.21 | <0.001 |
| | (1.70-3.15) | (2.40-4.35) | |

*Log-transformed before analysis. Data were presented as mean ± standard deviation or median (25th to 75th percentile). TSP2, thrombospondin 2; BMI, body mass index; WC waist circumference; BP, blood pressure; NAFLD, non-alcoholic fatty liver disease; GLP1rA, glucagon-like peptide 1 receptor agonists; SGLT2i, sodium-glucose co-transporter 2 inhibitors; HbA1c, glycated hemoglobin; HDL-C, high-density lipoprotein cholesterol; LDL-C, low-density lipoprotein cholesterol; TG, triglycerides; ALT, alanine aminotransferase; AST, aspartate transaminase; eGFR, estimated glomerular filtration rate; CAP, controlled attenuation parameter; NFS, non-alcoholic fatty liver disease fibrosis score; FIB4, Fibrosis-4 index. Albuminuria was defined as urine albumin to creatinine ratio ≥30 mg/g; Conversion factors for HDL/LDL-C from mmol/l to mg/dL x38.9; TG from mmol/l to mg/dL x88.2.

In multivariable logistic regression analysis consisting of age, BMI, serum HDL-C, ALT, AST, platelet, CAP and TSP2 levels at baseline, it was found that serum TSP2 level at baseline was independently associated with development of ≥F3 fibrosis (OR 2.82, 95% CI 1.37-5.78, p=0.005), together with baseline BMI (OR 1.12, 95% CI 1.03-1.21, p=0.007), platelet count (OR 0.992, 95% CI 0.987-0.998, p=0.01) and CAP values (OR 1.02, 95% CI 1.01-1.03, p<0.001) (Table 6).

TABLE 6
Multivariable logistic regression analysis showing
the association of baseline serum TSP2 level
with hepatic fibrosis progression (N = 491)

| | |
|---|---|
| Baseline | Model |

| variable | OR (95% CI) | p-value |
|---|---|---|
| BMI, kg/m² | 1.12 | 0.007 |
| | (1.03-1.21) | |
| Platelets, ×10\hat{ }9/L | 0.992 | 0.010 |
| | (0.987-0.998) | |
| CAP, dB/m | 1.02 | <0.001 |
| | (1.01-1.03) | |
| TSP2*, ng/ml | 2.82 | 0.005 |
| | (1.37-5.78) | |

*Log-transformed before analysis.

Variables Included in the Analysis Consisted of Age, BMI, HDL-C, ALT, AST, Platelet Count, CAP and TSP2 Levels. Model Selection was Based on Akaike Information Criteria.

TSP2, thrombospondin 2; BMI, body mass index; HDL-C, high-density lipoprotein cholesterol; ALT, alanine aminotransferase; AST, aspartate aminotransferase; CAP, controlled attenuation parameter; OR, odds ratio; 95% CI, 95% confidence interval.

Although the AUROC to predict the development of ≥F3 fibrosis did not change significantly with the inclusion of baseline circulating TSP2 level (0.837 vs. 0.816, p=0.19 for the model with and without TSP2, respectively), there were significant improvements in both the NRI (37.3, 95% CI 6.4-68.1, p=0.02) and IDI (2.2, 95% CI 0.1-4.4, p=0.045) after the addition of baseline circulating TSP2 level to the clinical model consisting of BMI, platelet count and CAP values at baseline.

This study demonstrated the clinical relevance of circulating TSP2 level as a fibrosis biomarker in NAFLD, based on both cross-sectional and prospective approaches. It was observed that serum TSP2 level was associated with the presence of ≥F3 fibrosis, and was also an independent predictor of the development of advanced fibrosis in patients comorbid with NAFLD and type 2 diabetes.

The gene for TSP2, THBS2, was identified among the five prioritized genes specifically associated with liver fibrosis independent of etiology, and its expression was positively associated with stages of liver fibrosis. In mice treated with carbon tetrachloride (CCl4) and rats with bile duct ligation, which were both rodent models of liver fibrosis, hepatic TSP2 protein expression was found to be increased (Chen et al., Am J Physiol Gastrointest Liver Physiol; 316:G744-G754 (2019)). Similarly, in another study using Apolipoprotein E knockout (ApoE KO) mice fed with high fat high cholesterol diet, hepatic THBS2 gene expression was also higher in mice with severe fibrosis than those with mild fibrosis. In a study evaluating eight NAFLD patients with liver biopsy tissues available, significant up-regulation of the THBS2 gene expression in the liver was observed in patients with ≥F3 fibrosis compared to those with FO/1 fibrosis (Lou et al., Sci Rep; 7:4748 (2017)).

However, the mechanism to account for the raised circulating TSP2 levels in patients with advanced fibrosis appears not straightforward. Although both TSP1 and TSP2 are matricellular proteins involved in wound healing and remodeling processes, there are spatial and temporal differences in their tissue expression. In wound healing, for instance, it has been suggested that TSP1 might serve more as an acute phase reactant, while TSP2, which is primarily produced by fibroblasts, is more responsible for the subsequent remodeling processes. Liver fibrosis is also a wound healing and remodeling process secondary to chronic hepatic injury, such as in NAFLD. However, unlike TSP1 which activates latent transforming growth factor beta (TGF-β), a classical fibrogenic cytokine, TSP2 has minimal influence on TGF-ρ activity (Daniel et al., J Am Soc Nephrol; 18:788-798 (2007)). In an experimental model of glomerulonephritis, although genetic ablation of TSP2 in mice accelerated endothelial cell proliferation and capillary repair after renal injury, it also resulted in heightened inflammation, matrix accumulation and increased glomerulosclerosis compared to wild-type mice. Similarly, in another rodent study of experimental brain injury, the lack of TSP2 had also been shown to dampen the recovery of blood brain barrier and prolong neuro-inflammation after foreign body implantation in mice, with elevated local production of matrix metalloproteinase (MMP)-2 and MMP-9 levels, (Tian et al., Am J Pathol; 179:860-868 (2011)), both of which were also implicated in the pathogenesis of liver fibrosis. Moreover, in rheumatoid arthritis, high TSP2 expression was found to be produced by synovial fibroblasts, endothelial cells and macrophages in patients with diffuse arthritis. However, in vivo models of human rheumatoid arthritis demonstrated that over-expression of TSP2 in fact resulted in marked reduction in lesion vascularization, tissue-infiltrating T-cell density, as well as the production of pro-inflammatory mediators including tumour necrosis factor alpha (TNFα) and interferon-gamma (IFN-γ) (Park et al., Am J Pathol; 165:2087-2098 (2004)). On the other hand, in a recent in vitro study, THBS2 mRNA was found to be highly expressed in hepatic stellate cells, and over-expression of THBS2 significantly promoted their activation. In diabetes, previous studies had also suggested TSP2 as a biomarker of proliferative diabetic retinopathy (PDR), as its levels in the vitreous fluid were significantly up-regulated in patients with PDR and in those with active neovascularization (Abu El-Asrar. Acta Ophthalmol; 91:e169-177 (2013)). The authors proposed that myofibroblasts might augment TSP2 secretion to protect the tissues from excessive angiogenesis in PDR. Recently, it was found that TSP2 expression in the skin was significantly increased in patients with type 2 diabetes to almost 3-fold that of individuals without diabetes. In vitro analysis revealed that hyperglycemia could activate the hexosamine pathway and nuclear factor kappa B (NFκB) signaling, thereby increasing TSP2 expression in fibroblasts, although it has also been shown previously that hyperglycemia could also up-regulate TSP2 expression through increased oxidative stress (Bae et al., Arterioscler Thromb Vasc Biol; 33:1920-1927 (2013)). Collectively, whether the effect of TSP2 on fibrogenesis is tissue-specific, or the up-regulation of hepatic and circulating TSP2 levels in patients with advanced fibrosis represents a compensatory response to underlying inflammation and oxidative stress in NASH, requires evaluation in further mechanistic studies.

There were several limitations in this study. First, the observational study design did not allow for any causal relationship to be inferred between high circulating TSP2 level and the development of ≥F3 fibrosis in patients with type 2 diabetes. Second, liver biopsy was not performed. However, VCTE has been increasingly utilized as an accurate alternative tool for evaluation of hepatic fibrosis in NAFLD, with an AUROC of up to 0.93 for the detection of biopsy-proven fibrosis (Castera et al., Gastroenterology; 156:1264-1281 e1264 (2019)). This is especially relevant in the assessment of a large number of stable and asymptomatic patients comorbid with type 2 diabetes and NAFLD, or MAFLD (Eslam et al., J Hepatol; 73:202-209 (2020)). Lastly, the median observational period was only 1.5 years, which could have contributed to the relatively low event rate of fibrosis progression as compared to previous studies.

The present study shows evidence for employing circulating TSP2 level as a biomarker of advanced fibrosis, which will be useful for hepatic risk stratification among the large number of patients comorbid with type 2 diabetes and NAFLD. Notably, with its high NPV of ≥90% and the significant improvement of AUROC to identify ≥F3 fibrosis on VTCE, this will be especially useful in diabetes clinics where VCTE is not readily available. Patients with high circulating TSP2 level, which indicates a greater risk of harboring ≥F3 fibrosis and fibrosis progression on VCTE, can be identified for referrals to hepatologists for further evaluation. Moreover, these patients can be prioritized for anti-diabetic agents that could improve hepatic fibrosis, liver dysfunction and/or steatosis, as well as new NAFLD treatments when clinically available.

Significantly, existing detection and diagnosis of NAFLD generally requires biopsy and histological analysis. For example, histological changes and NAFLD activity scores can only be determined using liver biopsy. Their invention was based on cross-sectional and prospective studies. The present study involved individuals with type 2 diabetes mellitus and used both cross-sectional histology and prospective approaches and demonstrated that serum TSP2 level is effective as a marker of ≥F3 fibrosis on vibration controlled transient elastography (VCTE). As described herein, this allows detection and diagnosis of ≥F3 fibrosis in NAFLD without the need for liver biopsy or histological analysis.

1. A method of non-invasively detecting advanced liver fibrosis in a subject with non-alcoholic fatty liver disease (NAFLD), the method comprising:
(a) measuring circulating levels of a biomarker thrombospondin 2 (TSP2) in a sample from the subject, and
(b) detecting advanced liver fibrosis when the circulating levels of TSP2 in the sample from the subject are greater than about 3.6 ng/ml.

2. The method of claim 1, wherein measuring comprises contacting the sample from the subject with a set of capture reagents, wherein the set of capture reagents comprises one or more antibody binding fragments having a binding specificity to TSP2 and no binding specificity to TSP1, TSP3, TSP4 and TSP5.

3. (canceled)

4. The method of a claim 1, wherein measuring comprises contacting the sample from the subject with a set of capture reagents comprising two antibody binding fragments, each having a binding specificity to distinct sites of TSP2.

5. The method of claim 1, wherein the TSP2 is human TSP2.

6. The method of claim 1, wherein the sample is diluted or undiluted blood or serum, diluted at a ratio between about 1:5 and 1:500 (v/v) of the sample to a sample dilution buffer.

7. (canceled)

8. The method of claim 1, wherein advanced liver fibrosis comprises fibrosis about or over grade F3 as measured by vibration controlled transient elastography (VCTE).

9. The method of claim 1, wherein advanced liver fibrosis comprises fibrosis of about or over grade F3 as graded by liver stiffness (LS) measurement cut off value of about 9.6 kiloPascal (kPa) on M probe or 9.3 kPa on XL probe, and greater.

10. The method of claim 1, wherein
advanced liver fibrosis is detected with a sensitivity of about or greater than 80% and specificity of about or greater than 60% when the circulating levels of TSP2 in the sample from the subject are greater than about 3.6 ng/ml.

11. The method of claim 1, wherein
advanced liver fibrosis is detected with negative predictive value of about or over 90%.

12. The method of claim 1, wherein
measuring is by an immunoassay having a lowest detection limit for TSP2 between about 0.01 ng/ml and about 0.18 ng/ml.

13. The method of claim 1, wherein the subject has NAFLD and one or more of the diseases selected from the group consisting of metabolic syndrome, type 2 diabetes mellitus, cardiovascular disease (CVD), and chronic kidney disease (CKD).

14. A method of detecting risk of developing of advanced liver fibrosis in a subject with non-alcoholic fatty liver disease (NAFLD), the method comprising:
(a) measuring circulating levels of a biomarker thrombospondin 2 (TSP2) in a sample from the subject, and
(b) detecting risk of developing of advanced liver fibrosis per unit increase in the log-transformed circulating level of TSP2 in the sample from the subject measured in ng/ml.

15. The method of claim 14, wherein the method detects a 2.82 times greater risk of developing advanced liver fibrosis per unit increase in log-transformed circulating level of TSP2 in the sample from the subject.

16. The method of claim 14, wherein detecting the risk of developing of advanced liver fibrosis is for risk within a period between about 0.1 years and about 3 years from step (a).

17. The method of claim 14, wherein measuring comprises contacting the sample from the subject with a set of capture reagents, wherein the set of capture reagents comprises one or more antibody binding fragments having a binding specificity to TSP2 and no binding specificity to TSP1, TSP3, TSP4 and TSP5.

18. (canceled)

19. The method of claim 14, wherein measuring comprises contacting the sample from the subject with a set of capture reagents comprising two antibody binding fragments, each having a binding specificity to distinct sites of TSP2.

20. The method of claim 14, wherein TSP2 is human TSP2.

21. The method of claim 14, wherein the sample is diluted or undiluted blood or serum, diluted at a ratio between about 1:5 and 1:500 (v/v) of the sample to a sample dilution buffer.

22. (canceled)

23. The method of claim 14, wherein the subject has NAFLD and one or more of the diseases selected from the group consisting of metabolic syndrome, type 2 diabetes mellitus, cardiovascular disease (CVD), and chronic kidney disease (CKD).

24. The method of claim 1 comprising identifying the subject as in need of liver fibrosis treatment, treating the subject to reduce liver fibrosis, or both identifying the subject as in need of liver fibrosis treatment and treating the subject to reduce liver fibrosis.

25. (canceled)

26. A kit comprising a set of capture reagents for use in the method of claim 1.
